(19) United States
(12) Patent Application Publication
(10) Pub. No.: US 2023/0398295 A1
(43) Pub. Date: Dec. 14, 2023

(54) HEMODYNAMIC MANAGEMENT SYSTEM, APPARATUS, AND METHODS

(71) Applicants: BAXTER INTERNATIONAL INC., Deerfield, IL (US); BAXTER HEALTHCARE SA, Glattpark (Opfikon) (CH)

(72) Inventors: Kelsey Veserat, Madison, WI (US); Jessica Tam, Chicago, IL (US)

(21) Appl. No.: 18/198,987

(22) Filed: May 18, 2023

Related U.S. Application Data
(60) Provisional application No. 63/343,222, filed on May 18, 2022.

(51) Int. Cl. A61M 5/172; G16H 10/60; G16H 70/40; G16H 20/17

(52) U.S. Cl. CPC A61M 5/1723; G16H 10/60; A61M 2230/04; G16H 20/17; G16H 70/40

(57) ABSTRACT

A hemodynamic management system, apparatus, and method are disclosed herein. An example hemodynamic management apparatus includes a display interface screen and a processor configured to access a patient medical record using a patient identifier and determine, from the patient medical record, a new infusion start event associated with an infusion pump that is fluidly connected to a patient. The processor also causes the display interface screen to display an infusion line mapping interface that shows a graphical illustration of a human body and potential access sites, prompt for selection of an access site within the infusion line mapping interface, and after receiving a selection of an access site, associate an infusion pump identifier and the selected access site. The processor also causes the display interface screen to display information from the new infusion start event in conjunction with the selected access site shown within the infusion line mapping interface.

### PRIORITY CLAIM

This application claims priority to and the benefit as a non-provisional application of U.S. Provisional Patent Application No. 63/343,222, filed May 18, 2022, the entire contents of which are hereby incorporated by reference and relied upon.

### BACKGROUND

Hemodynamics refers the fluid dynamics of blood flow in a patient. In a normal circulatory system, the volume of blood returning to a patient's heart is approximately equal to the volume that is pumped. Hemodynamic monitoring detects when there is an imbalance between the volume of blood pumped and the volume of blood returned to a patient's heart. This monitoring is important to diagnose cardiovascular disorders, post-operation trauma, sepsis, respiratory failure, and neurological injuries. Hemodynamic health is determined through a number of patient parameters including blood pressure or mean arterial pressure (“MAP”), heart rate, arterial oxygen saturation (“SpO2”), body temperature, and/or fluid responsiveness. Other parameters of hemodynamic health that may be monitored or calculated include blood flow velocity, cardiac output (“CO”), arterial pressure, peripheral, total, or systemic vascular resistance (“SVR”), turbulence, wall tension, stroke volume (“SV”), stroke volume variability (“SVV”), and pulse pressure variation (“PPV”).

In hemodynamically unstable patients, the volume of blood flow entering/leaving the heart becomes unbalanced. This can result in a dramatic increase or decrease in blood pressure. This can also result in an elevated or low heart rate, sepsis, or even respiratory issues. In many instances, hemodynamic instability during treatments can lengthen or complicate recovery, lead to more serious health conditions, or even result in death. Accordingly, hemodynamic stability is oftentimes a critical goal for clinicians that work in intensive care units (“ICUs”).

While caring for hemodynamically unstable patients, clinicians are presented with a complex environment that does not facilitate the early detection of threats to favorable clinical outcomes. Oftentimes, clinicians struggle to access and interpret relevant data points for clinical decision making. Even when accessed, the relevant data includes disparate data points and can only be obtained through distracting and non-integrated point-of-care (“POC”) systems. Coupling this less than desirable data access situation with a shortage of skilled clinicians and a lack of standardization creates challenges in care coordination and delivery. This is especially true in regard to hemodynamic care where there are hectic, high stakes patient conditions that require rapid and accurate responses to address hemodynamic instability. Known systems require the use of highly inefficient and manual clinical tasks that must first be completed to hemodynamically stabilize a patient.

### SUMMARY

An example system, method, and apparatus are disclosed for hemodynamic management. The example system, method, and apparatus are configured to merge infusion therapy information with physiological and hemodynamic information into a single display device located at a patient's bedside. In some instances, the physiological information may include dialysis information or other information that is indicative of fluid removed from a patient. The system, method, and apparatus accordingly provide hemodynamic information regarding a net fluid balance of a patient in real time or near-real time.

The example system, method, and apparatus may also be configured to provide for a hemodynamic assessment, which is integrated with the infusion therapy information, physiological information, and hemodynamic information that is shown on the single display device. The hemodynamic assessment provides an indication of fluid responsiveness in a patient by inducing a reversible increase in cardiac preload. The display of information related to a hemodynamic assessment in conjunction with infusion therapy information, physiological information, and hemodynamic information provides a better clinician experience by consolidating critical patient information into one display device. Merging this information also enables the display device to execute one or more algorithms or protocols to provide automated clinical decision support. Compared to known electronic medical record (“EMR”) systems that are repositories of patient health information, the system, method, and apparatus disclosed herein are configured to provide real time or near-real time individualized hemodynamic-specific management and corresponding clinical decision support.

In some embodiments, the example system, method, and apparatus are configured to manage the mapping of one or more infusion therapies to intravenous infusion sites on a patient. The system, method, and apparatus use a drug library file that specifies drug-to-drug and/or drug-to-access site type incompatibilities to determine when a planned infusion therapy may be harmful to a patient. The system, method, and apparatus may generate an alert that indicates which drugs are incompatible and/or which drugs for an infusion site are incompatible. In some embodiments, the system, method, and apparatus may recommend one or more alternative infusion sites.

Aspects of the subject matter described herein may be useful alone or in combination with one or more other aspect described herein. Without limiting the foregoing description, in a first aspect of the present disclosure, a hemodynamic management apparatus includes a display interface screen, a memory device storing a patient identifier, and a processor communicatively coupled to the display interface screen and the memory device. The processor is configured to access a patient medical record within an electronic medical record database using the patient identifier and determine, from the patient medical record, a new infusion start event associated with an infusion pump that is fluidly connected to a patient corresponding to the patient medical record. The new infusion start event includes information indicative of an infusion pump identifier, an infused fluid name, an infusion rate, a volume to be infused, a dose, a volume remaining, and a time the new infusion start event was generated by the infusion pump. The processor is also configured to cause the display interface screen to display an infusion line mapping interface that shows a graphical illustration of a human body and potential access sites and prompt for selection of an access site within the infusion line mapping interface. The processor is further configured to, after receiving a selection of an access site, associate within the memory device the infusion pump identifier and the selected access site. Additionally, the processor is configured to cause the display interface screen to display at least some of the information associated with the new infusion start event in conjunction with the selected access site shown within the infusion line mapping interface.

In accordance with a second aspect of the present disclosure, which may be used in combination with the first aspect, the memory device is configured to store a drug library that specifies fluid type-to-access site type incompatibilities. In addition, the processor is further configured to, after receiving the selection of the access site, perform a check for a fluid type-to-access site incompatibility between the selected access site and the infused fluid name, and when there is an incompatibility display an alert within the infusion line mapping interface and provide a prompt to change an access site for the infused fluid name associated with the infusion pump, remove the alert after receiving a selection of a second access site, and display the at least some of the information associated with the new infusion start event in conjunction with the selected second access site.

In accordance with a third aspect of the present disclosure, which may be used in combination with any one or more of the preceding aspects, the processor is further configured to use the drug library to determine a compatible access site based on the infused fluid name, and cause the infusion line mapping interface to display a recommendation to fluidly connect the infusion pump to the determined, compatible access site.

In accordance with a fourth aspect of the present disclosure, which may be used in combination with any one or more of the preceding aspects, the processor is further configured to determine, from the patient medical record, a second new infusion start event associated with a second infusion pump that is fluidly connected to the patient corresponding to the patient medical record. The second new infusion start event includes second information indicative of a second infusion pump identifier, a second infused fluid name, a second dose, a second infusion rate, a second volume to be infused, a second volume remaining, and a second time the second new infusion start event was generated by the second infusion pump. The processor is also configured to cause the display interface screen to display the infusion line mapping interface, prompt for selection of a second access site within the infusion line mapping interface, and after receiving a selection of the second access site, cause the display interface screen to display at least some of the second information associated with the second new infusion start event in conjunction with the selected second access site.

In accordance with a fifth aspect of the present disclosure, which may be used in combination with any one or more of the preceding aspects, the memory device is configured to store a drug library that specifies fluid type-to-fluid type incompatibilities for access sites. In addition, the processor is further configured to, after receiving the selection of the second access site, perform a check for a fluid type-to-fluid type incompatibility between the infused fluid name and the second infused fluid name, and when there is an incompatibility display an alert within the infusion line mapping interface and provide a prompt to change an access site for the second infused fluid name associated with the second infusion pump, remove the alert after receiving a selection of a third access site, and display at least some of the second information associated with the second new infusion start event in conjunction with the selected third access site.

In accordance with a sixth aspect of the present disclosure, which may be used in combination with any one or more of the preceding aspects, the processor is further configured to receive, from at least one sensor, hemodynamic information indicative of a cardiac stroke volume, a cardiac output, a cardiac index, a heart rate, a total peripheral resistance index (“TPRI”), or fluid responsiveness, and cause at least some of the hemodynamic information to be displayed in conjunction with the infusion line mapping interface or within a separate hemodynamic interface.

In accordance with a seventh aspect of the present disclosure, which may be used in combination with any one or more of the preceding aspects, the processor is further configured to determine hemodynamic information indicative of a cardiac stroke volume, a cardiac output, a cardiac index, a heart rate, a total peripheral resistance index (“TPRI”), or fluid responsiveness, and cause at least some of the hemodynamic information to be displayed in conjunction with the infusion line mapping interface or within a separate hemodynamic interface.

In accordance with an eighth aspect of the present disclosure, which may be used in combination with any one or more of the preceding aspects, the processor is further configured to combine the infusion rate of the infused fluid name with other infusion rates associated with the patient that are specified in the patient medical record, determine fluid output rates that are specified in the patient medical record, the fluid output rates corresponding to at least one of dialysis, urine monitoring, or a fluid drain, determine a fluid balance as a difference between the combined infusion rates and the combined fluid output rates, and display at least the fluid balance within an interactive graphical interface or the infusion line mapping interface that is shown by the display interface screen.

In accordance with a ninth aspect of the present disclosure, which may be used in combination with any one or more of the preceding aspects, the processor is further configured to determine, from the patient medical record, a new infusion event that specifies a time the new infusion event was generated by the infusion pump and is indicative of at least one of a changed infusion rate, a changed volume to be infused, or a changed volume remaining, and update the infusion line mapping interface based on information associated with the new infusion event.

In accordance with a tenth aspect of the present disclosure, which may be used in combination with any one or more of the preceding aspects, the processor is further configured to receive information indicative of an alarm event or determine, from the patient medical record, an alarm event, and display a graphic or at least some of the information that is indicative of the alarm event, where the alarm event includes at least one of information indicative of an infiltration detection, a line occlusion, or a fluid container being empty or near-empty.

In accordance with an eleventh aspect of the present disclosure, which may be used in combination with any one or more of the preceding aspects, the infusion line mapping interface is configured to display the infused fluid name and the infusion rate, and display a graphical icon that is indicative of the volume remaining or a time indicative of the volume remaining.

In accordance with a twelfth aspect of the present disclosure, which may be used in combination with any one or more of the preceding aspects, the processor is further configured to determine the volume remaining or a time indicative of the volume remaining is less than a threshold, display the volume remaining or the time indicative of the volume remaining in conjunction with the graphical icon, and change a color of the graphical icon and the selected access site.

In accordance with a thirteenth aspect of the present disclosure, which may be used in combination with any one or more of the preceding aspects, the selection of the graphical icon causes the processor to display at least the infusion pump identifier, the volume to be infused, the infusion rate, and the infused fluid name.

In accordance with a fourteenth aspect of the present disclosure, which may be used in combination with any one or more of the preceding aspects, the selection of the graphical icon causes the processor to transmit a message causing the infusion pump to generate a sound or provide a visual indication.

In accordance with a fifteenth aspect of the present disclosure, which may be used in combination with any one or more of the preceding aspects, the patient identifier is entered into the display interface screen and stored to the memory device or determined from the patient medical record.

In accordance with a sixteenth aspect of the present disclosure, which may be used in combination with any one or more of the preceding aspects, the apparatus further includes an adapter for connection to a hub device that is also connected to the infusion pump.

In accordance with a seventeenth aspect of the present disclosure, which may be used in combination with any one or more of the preceding aspects, a hemodynamic management method includes accessing, via a processor, a patient medical record within an electronic medical record database using a patient identifier, and determining, from the patient medical record using the processor, a new infusion start event associated with an infusion pump that is fluidly connected to a patient corresponding to the patient medical record. The new infusion start event includes information indicative of an infusion pump identifier, an infused fluid name, an infusion rate, a volume to be infused, a dose, a volume remaining, and a time the new infusion start event was generated by the infusion pump. The method further includes causing, via the processor, a display interface screen to display an infusion line mapping interface that shows a graphical illustration of a human body and potential access sites, prompting, via the processor, for selection of an access site within the infusion line mapping interface, and after receiving a selection of an access site, associating, via the processor, the infusion pump identifier and the selected access site. The method additionally includes causing, via the processor, the display interface screen to display at least some of the information associated with the new infusion start event in conjunction with the selected access site shown within the infusion line mapping interface.

In accordance with an eighteenth aspect of the present disclosure, which may be used in combination with any one or more of the preceding aspects, the method further includes after receiving the selection of the access site, performing, via the processor, a check for a fluid type-to-access site incompatibility between the selected access site and the infused fluid name using a drug library that specifies fluid type-to-access site type incompatibilities, and when there is an incompatibility displaying, via the processor, an alert within the infusion line mapping interface and providing a prompt to change an access site for the infused fluid name associated with the infusion pump, removing, via the processor, the alert after receiving a selection of a second access site, and displaying, via the processor, the at least some of the information associated with the new infusion start event in conjunction with the selected second access site.

In accordance with a nineteenth aspect of the present disclosure, which may be used in combination with any one or more of the preceding aspects, the method further includes determining, via the processor, a compatible access site based on the infused fluid name using the drug library, and causing, via the processor, the infusion line mapping interface to display a recommendation to fluidly connect the infusion pump to the compatible access site.

In accordance with a twentieth aspect of the present disclosure, which may be used in combination with any one or more of the preceding aspects, the method further includes combining, via the processor, the infusion rate of the infused fluid name with other infusion rates associated with the patient that are specified in the patient medical record, determining, via the processor, fluid output rates that are specified in the patient medical record, the fluid output rates corresponding to at least one of dialysis, urine monitoring, or a fluid drain, determining, via the processor, a fluid balance as a difference between the combined infusion rates and the combined fluid output rates, and displaying, via the processor, at least the fluid balance within an interactive graphical interface or the infusion line mapping interface that is shown by the display interface screen.

In accordance with a twenty-first aspect of the present disclosure, any of the structure and functionality illustrated and described in connection with FIGS. 1 to 24 may be used in combination with any of the structure and functionality illustrated and described in connection with any of the other of FIGS. 1 to 24 and with any one or more of the preceding aspects.

In light of the present disclosure and the above aspects, it is therefore an advantage of the present disclosure to provide a hemodynamic management apparatus and system that displays in real-time hemodynamic parameters for providing a hemodynamic assessment.

It is another advantage of the present disclosure to provide infusion line management by showing current infusions relative to patient access sites within a single display interface.

It is a further advantage of the present disclosure to provide fluid type-to-fluid type and access site compatibility checking before a new infusion can proceed.

It is yet a further advantage of the present disclosure to provide automated fluid balance monitoring.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a diagram of a hemodynamic management system including a hemodynamic management apparatus, according to an example embodiment of the present disclosure.

FIG. 2 is a diagram that shows one example of sensors communicating directly with the hemodynamic management apparatus of FIG. 1, according to an example embodiment of the present disclosure.

FIG. 3 is another diagram of the hemodynamic management system of FIG. 1, according to another example embodiment of the present disclosure.

FIG. 4 is a diagram of a hub device configured to connect to the hemodynamic management apparatus of FIGS. 1 to 3, according to an example embodiment of the present disclosure.

FIG. 5 is another diagram of the hub device of FIG. 4 with the hemodynamic management apparatus placed adjacent to a communication module, according to an example embodiment of the present disclosure.

FIG. 6 is another diagram of the hub device with a slim-version of the hemodynamic management apparatus, according to an example embodiment of the present disclosure.

FIG. 7 is a diagram of the hemodynamic management apparatus connected to a pole, according to an example embodiment of the present disclosure.

FIG. 8 shows a flow diagram illustrating an example procedure to configure the hemodynamic management apparatus of FIGS. 1 to 7, according to an example embodiment of the present disclosure.

FIG. 9 is a diagram of a registration interface for patient association that is displayed by the hemodynamic management apparatus of FIGS. 1 to 7, according to an example embodiment of the present disclosure.

FIG. 10 shows a flow diagram illustrating an example procedure for infusion line management performed by the hemodynamic management apparatus of FIGS. 1 to 7, according to an example embodiment of the present disclosure.

FIG. 11 is a diagram of an example infusion line mapping interface that may be displayed by a display interface screen of the hemodynamic management apparatus of FIGS. 1 to 7, according to an example embodiment of the present disclosure.

FIG. 12 is a diagram of the infusion line mapping interface of FIG. 11 showing an incompatibility between two infusions connected to the same access site, according to an example embodiment of the present disclosure.

FIG. 13 is a diagram that shows different possible physiological data that is input into the hemodynamic management apparatus of FIGS. 1 to 7, according to an example embodiment of the present disclosure.

FIG. 14 is a diagram of a hemodynamic dashboard interface that is displayed on a display interface screen by an application of the hemodynamic management apparatus of FIGS. 1 to 7, according to an example embodiment of the present disclosure.

FIG. 15 shows diagrams of fluid responsiveness interfaces that may be displayed by the application of the hemodynamic management apparatus of FIGS. 1 to 7, according to an example embodiment of the present disclosure.

FIG. 16 shows the hemodynamic dashboard interface after a hemodynamic assessment has been performed, according to an example embodiment of the present disclosure.

FIG. 17 is a diagram of an assessment interface that shows a status of a bolus hemodynamic assessment, according to an example embodiment of the present disclosure.

FIG. 18 is a diagram of a fluid responsiveness interface that may be shown by the application of the hemodynamic management apparatus after a hemodynamic assessment has occurred or is ongoing, according to an example embodiment of the present disclosure.

FIG. 19 is a diagram of the hemodynamic dashboard interface of FIG. 14 with an infusion status section changed to indicate information indicative of an alarm or an alert, according to an example embodiment of the present disclosure.

FIG. 20 is a diagram of the infusion line mapping interface of FIG. 11 showing information indicative of an alarm or an alert regarding a bag near-empty, according to an example embodiment of the present disclosure.

FIG. 21 is a diagram of the hemodynamic dashboard interface of FIG. 14 displaying an indication of an alert related to a detection of infiltration, according to an example embodiment of the present disclosure.

FIG. 22 is a diagram of the infusion line mapping interface of FIG. 11 showing information indicative of an alarm or an alert regarding an infiltration detection, according to an example embodiment of the present disclosure.

FIG. 23 is a diagram of a fluid balance interface, according to an example embodiment of the present disclosure.

FIG. 24 is a diagram of a detailed net fluid balance interface, according to an example embodiment of the present disclosure.

### DETAILED DESCRIPTION

The present disclosure relates in general to a method, system, and apparatus for hemodynamic management. The method, system, and apparatus include a hemodynamic management device that is configured for use at a patient's bedside. The hemodynamic management device is configured to receive data from a plurality of medical devices. The data is compiled to provide a real time or near-real time hemodynamic assessment in conjunction with a total fluid balance for a patient. The hemodynamic assessment may be carried out via a passive leg raise (“PLR”), an infusion bolus, and/or an automatic detection of an infusion bolus. The hemodynamic assessment provides an indication of fluid responsiveness in a patient by inducing a reversible increase in cardiac preload.

As described herein, a total fluid balance is a net increase or decrease in patient fluid. The method, system, and apparatus are configured to aggregate data from medical devices to determine a cumulative fluid infusion rate and a cumulative fluid output rate. The cumulative fluid infusion rate and output rate are subtracted to determine a net fluid increase/decrease for a time period, such as a second, a minute, ten minutes, etc. The net fluid increase/decrease is trended over time to determine how much fluid a patient has lost or gained within a specified time period, such as an hour, four hours, eight hours, twelve hours, twenty-four hours, forty-eight hours, seventy-two hours, etc. Together, the total fluid balance and hemodynamic assessment provide critical information that enables the automatic or guided identification or prediction of patient hemodynamic instability. Such identification enables clinicians to more easily address hemodynamic instability at a detected or predicted onset before a patient's condition worsens.

The method, system, and apparatus disclosed herein are additionally configured to provide an automated mapping of fluid infusions. Some critically ill patients may have as many as fifteen simultaneous infusions. Each infusion is connected to a fluid container (such as an intravenous (“IV”) bag). A fluid from the fluid container is pumped by a respective infusion pump via IV tubing or an IV line. Separate IV lines may be connected to a patient at respective access sites. However, to reduce a number of access sites used (reducing patient discomfort), some IV lines may be connected together upstream from the access site location. Rather than having a clinician perform manual compatibility checking and infusion line tracking, the method, system, and apparatus automatically perform compatibility checks to ensure an infused fluid is compatible with a particular infusion access site or compatible with one or more other fluids at a particular infusion access site. The automated mapping also enables the method, system, and apparatus to provide a real time summary of a status of all the infusions within a single display. This significantly reduces a burden on a clinician from having to individually check many different infusions pumps.

Reference is made herein to infusion access sites, intravenous access sites, or more generally, access sites. As discussed below, an access site is a location on a patient that receives an infused fluid. Access sites are typically veins, which permit the infusion of a fluid directly into a patient's blood. For example, humans include numerous venous access sites including the internal jugular vein, the external jugular vein, the left subclavian vein, the superior vena cava vein, the cephalic vein, the basilica vein, the median cubital vein, the median cephalic vein, the median antebrachial vein, the accessory cephalic vein, the dorsal venous arch, metacarpal veins, and finger veins. However, it should be appreciated that the method, system, and apparatus disclosed herein may provide mapping to any patient access site.

As disclosed herein, hemodynamic management includes hemodynamic monitoring, vasoactive administration/titration, fluid resuscitation, and urine output/fluid balance tracking. The example method, system, and apparatus are configured to receive data from medical devices and one or more physiological sensors, and use this data for hemodynamic management. The method, system, and apparatus include instructions that specify which of the data is related to hemodynamic management for selection or filtering. The instructions may also specify certain calculations to perform to determine other hemodynamic parameters. The example method, system, and apparatus may also include one or more instructions or algorithms to monitor or analyze trends or relations between the hemodynamic parameters for identification of fluid balance variations, instability, and/or physiological changes. In some instances, the method, system, and apparatus may also include instructions or algorithms for predicting future hemodynamic instability in a patient by analyzing hemodynamic parameter trends or relations.

The example method, system, and apparatus disclosed herein accordingly provide clinicians with an immediate and complete picture of a patient's hemodynamic condition within a single display or interface. The display of all relevant hemodynamic parameters in one interface enables clinicians to make more informed decisions at the point-of-care through early prediction and detection of hemodynamic issues. The example system disclosed herein also simplifies and streamlines hospital workflow by reducing the number of information sources and interfaces that a clinician has to access to make an informed decision, thereby increasing a clinician's confidence and efficiency. Most importantly, the example system disclosed herein provides more effective management of hemodynamics for unstable patients in critical care that can lead to better outcomes and shorter lengths of stay in the ICU, or hospital more generally. It should be appreciated that the method, system, and apparatus disclosed herein may be applied to other areas, including electrolyte replacement and renal function monitoring. The example method, system, and apparatus may also be used for antibiotic management, blood glucose management, infection surveillance and line tracking, and/or wounds/drain management.

### Hemodynamic Management System

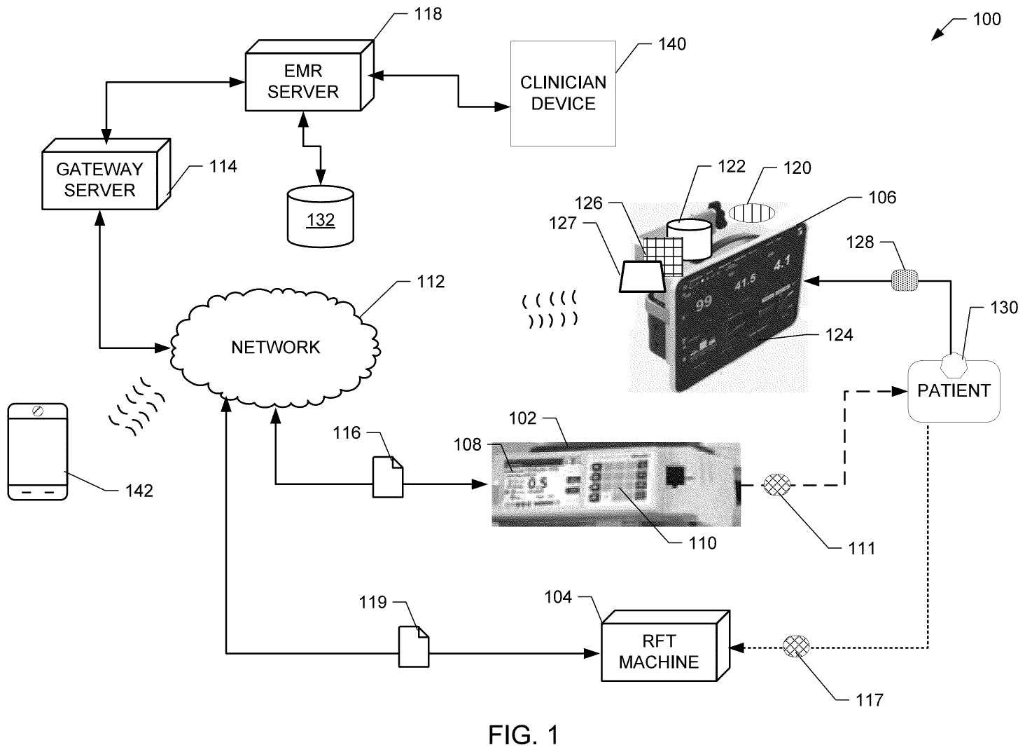

FIG. 1 is a diagram of a hemodynamic management system 100, according to an example embodiment of the present disclosure. The system 100 includes medical devices that operate in conjunction with a patient. The medical devices include, for example, an infusion pump 102 and a dialysis or renal failure therapy (“RFT”) machine 104. The system 100 also includes a hemodynamic management apparatus 106.

The example infusion pump 102 may include any pump capable of delivering an intravenous therapy to a patient via one or more intravenous line sets. Examples include a syringe pump, a linear peristaltic pump, a large volume parenteral pump, an ambulatory pump, multi-channel pump, etc. The example infusion pump 102 includes a display 108 and an interface 110 to enable a clinician to specify or program an infusion therapy. In addition to manual programming, the infusion pump 102 may receive electronic prescriptions from a hospital information system via a network 112 and a gateway server 114. The infusion pump 102 may include one or more drug libraries that include particular limits based on care area, dose, rate of change, drug type, concentration, patient age, patient weight, etc.

The infusion pump 102 is configured to perform an infusion therapy on a patient, which includes the infusion of one or more fluids, solutions, or drugs 111 into the patient. The infusion pump 102 operates according to an infusion prescription entered by a clinician at the user interface 110 of the pump or received via the gateway server 114. The infusion pump 102 may compare the prescription to the drug library and provide any alerts or alarms when a parameter of the prescription violates a soft or hard limit. The infusion pump 102 is configured to monitor the progress of the therapy and periodically transmit infusion therapy progress data 116 to the gateway server 114. The infusion therapy progress data 116 may include, for example, an infusion rate, a dose, a total volume infused, a time remaining for the therapy, a fluid concentration, a rate change, a volume remaining within a medication container, a fluid name, a patient identifier, titration information, bolus information, a care area identifier, a timestamp when the data was generated, an alarm condition, an alert condition, an event, etc. In some instances, the infusion therapy progress data 116 includes a new infusion start event including information indicative of an infusion pump identifier, an infused fluid name, an infusion rate, a volume to be infused, a dose, a volume remaining, and/or a time the new infusion start event was generated by the infusion pump. The infusion pump 102 may transmit the data 116 continuously, periodically (e.g., every 30 seconds, 1 minute, etc.), or upon request by the gateway server 114.

The example RFT machine 104 of FIG. 1 may include any hemodialysis, hemofiltration, hemodiafiltration, continuous renal replacement therapy (“CRRT”), or peritoneal dialysis machine. CRRT is a dialysis modality typically used to treat critically ill, hospitalized patients in an intensive care unit who develop acute kidney injury (“AKI”). Unlike chronic kidney disease, which occurs slowly over time, AKI often occurs in hospitalized patients and typically occurs over a few hours to a few days. A patient, undergoing hemodialysis, for example, is connected to the RFT machine 104, where the patient's blood is pumped through the machine. The blood passes through a dialyzer of the machine 104, which removes waste, toxins and excess water (e.g., ultrafiltrate) 117 from the blood. The cleaned blood is returned to the patient.

Hemodialysis is a renal failure treatment in which waste from the blood is diffused across a semi-permeable membrane. During hemodialysis, blood is removed from the patient and flows through a semi-permeable membrane assembly (dialyzer), where the blood flows generally counter-current to a dialysis solution flowing on the other side of the semipermeable membrane. In the dialyzer, toxins from the blood travel across the semi-permeable membrane and exit the dialyzer into used dialysis solution (dialysate). The cleaned blood, having flowed through the dialyzer, is then returned to the patient.

In the dialyzer, a pressure differential is created across the semi-permeable membrane by removing dialysate at a flow rate that is greater than that used to introduce the dialysis solution into the dialyzer. This pressure differential pulls fluid containing small, middle, and large molecule toxins across the semi-permeable membrane. Flow and volume measurements are used to control the amount of fluid (ultrafiltration) that is removed. As illustrated above, a hemodialysis machine's pump typically pulls blood from the arterial side of the patient, pushes it into and through the dialyzer, and through a drip chamber that separates out air before returning the dialyzed blood to the venous side of the patient.

The RFT machine 104 can alternatively be a hemofiltration machine. Hemofiltration is another renal failure treatment, similar to hemodialysis. During hemofiltration, a patient's blood is also passed through a semipermeable membrane (a hemofilter), where fluid (including waste products) is pulled across the semipermeable membrane by a pressure differential. This convective flow brings certain sizes of molecular toxins and electrolytes (which are difficult for hemodialysis to clean) across the semipermeable membrane. During hemofiltration, a replacement fluid is added to the blood to replace fluid volume and electrolytes removed from the blood through the hemofilter. Hemofiltration in which replacement fluid is added to the blood prior to the hemofilter is known as pre-dilution hemofiltration. Hemofiltration in which replacement fluid is added to the blood after the hemofilter is known as post-dilution hemofiltration.

The RFT machine 104 can alternatively be a hemodiafiltration machine. Hemodiafiltration is a further renal failure treatment that uses hemodialysis in combination with hemofiltration. Blood is again pumped through a dialyzer, which accepts fresh dialysis fluid unlike a hemofilter. With hemodiafiltration, however, replacement fluid is delivered to the blood circuit, like with hemofiltration. Hemodiafiltration is accordingly a neighbor of hemodialysis and hemofiltration.

The RFT machine 104 can alternatively be a peritoneal dialysis machine. Peritoneal dialysis uses a dialysis solution, also called dialysate, which is infused into a patient's peritoneal cavity via a catheter. The dialysate contacts the peritoneal membrane of the patient's peritoneal cavity. Waste, toxins, and excess water pass from the patient's bloodstream, through the peritoneal membrane and into the dialysate due an osmotic gradient created by the solution. The spent dialysate is drained from the patient, removing waste, toxins, and excess water from the patient. This cycle is repeated.

The example peritoneal dialysis machine 104 may perform various types of additional peritoneal dialysis therapies, including continuous cycling peritoneal dialysis (“CCPD”), tidal flow automated peritoneal dialysis (“APD”), and continuous flow peritoneal dialysis (“CFPD”). APD machines perform drain, fill, and dwell cycles automatically, typically while the patient sleeps. APD machines free patients or clinicians from having to manually perform the treatment cycles and from having to transport supplies during the day. APD machines connect fluidly to an implanted catheter, to a source or bag of fresh dialysate, and to a fluid drain. APD machines pump fresh dialysate from a dialysate source, through the catheter, into the patient's peritoneal cavity, and allow the dialysate to dwell within the cavity, and allow the transfer of waste, toxins, and excess water to take place. The source can be multiple sterile dialysate solution bags. APD machines pump spent dialysate from the peritoneal cavity, though the catheter, to the drain. As with the manual process, several drain, fill, and dwell cycles occur during APD. A “last fill” occurs at the end of CAPD and APD, which remains in the peritoneal cavity of the patient until the next treatment.

CCPD treatments attempt to drain the patient fully upon each drain. CCPD and/or APD may be batch type systems that send spent dialysis fluid to a drain. Tidal flow systems are modified batch systems. With tidal flow, instead of removing all of the fluid from the patient over a longer period of time, a portion of the fluid is removed and replaced after smaller increments of time.

Peritoneal dialysis dialysate may include a solution or mixture that includes between 0.5% and 10% dextrose (or more generally glucose), preferably between 1.5% and 4.25%. Peritoneal dialysis dialysate may include, for example, Dianeal®, Physioneal®, Nutrineal®, and Extraneal® dialysates. The dialysate may additionally or alternatively include a percentage of icodextrin. It should be appreciated that in some embodiments of the present disclosure the dialysate may be infused into the patient via the infusion pump 102 rather than the RFT machine 104.

Continuous flow, or CFPD, dialysis systems clean or regenerate spent dialysate instead of discarding it. CFPD systems pump fluid into and out of the patient, through a loop. Dialysate flows into the peritoneal cavity through one catheter lumen and out another catheter lumen. The fluid exiting the patient passes through a reconstitution device that removes waste from the dialysate, e.g., via a urea removal column that employs urease to enzymatically convert urea into ammonia (e.g. ammonium cation). The ammonia is then removed from the dialysate by adsorption prior to reintroduction of the dialysate into the peritoneal cavity. Additional sensors are employed to monitor the removal of ammonia. CFPD systems are typically more complicated than batch systems.

In both hemodialysis and peritoneal dialysis, “sorbent” technology can be used to remove uremic toxins from waste dialysate, re-inject therapeutic agents (such as ions and/or glucose) into the treated fluid, and reuse that fluid to continue the dialysis of the patient. One commonly used sorbent is made from zirconium phosphate, which is used to remove ammonia generated from the hydrolysis of urea. Typically, a large quantity of sorbent is necessary to remove the ammonia generated during dialysis treatments.

Similar to the infusion pump 102, the RFT machine 104 may be programmed locally with a dialysis prescription or receive a dialysis prescription via the gateway server 114. The RFT machine 104 is configured to perform a dialysis therapy on a patient, which includes removing ultrafiltration from the patient. With peritoneal dialysis, the RFT machine 104 infuses dialysate into the patient during the fill cycles. For any dialysis prescription, the RFT machine 104 may compare parameters of the prescription to one or more limits and provide any alerts or alarms when a parameter of the prescription violates a soft or hard limit. The RFT machine 104 is configured to monitor the progress of the therapy and periodically transmit dialysis therapy progress data 119 to the gateway server 114. The dialysis therapy progress data 119 may include, for example, a fill rate, a dwell time, a drain or fluid removal rate, a blood flow rate, an effluent dose, an ultrafiltration removal rate, a dialysate removal rate, a total dialysate infused, a dialysate flow, a replacement pre-flow, a replacement post-flow, a patient weight balance, a return pressure, an excess patient fluid sign, a filtration fraction, a time remaining, a dialysate concentration, a dialysate name, a patient identifier, a room identifier, a care area identifier, a timestamp when the data was generated, an alarm condition, an alert condition, an event, etc. The RFT machine 104 may transmit the data continuously, periodically (e.g., every 30 seconds, 1 minutes, etc.), or upon request by the gateway server 114.

The hemodynamic management apparatus 106 includes a processor 120, a memory device 122, and a display interface screen 124. The memory device 122 stores instructions that specify or define operations performed by an application 126. Execution of the instructions by the processor 120 cause the hemodynamic management apparatus 106 to perform the actions discussed herein via the application 126. The processor 120 may include a controller, a logic device, an application specific integrated circuit (“ASIC”), a microcontroller, etc. The memory device 122 may include a flash drive, a solid state drive, or a hard disk drive.

The display interface screen 124 of the hemodynamic management apparatus 106 is configured to display information relevant to hemodynamic monitoring and management. As discussed in further detail below, this includes fluid balance information, hemodynamic assessment information, hemodynamic parameters, and/or alerts related to infiltration, infusion line occlusions, and/or a fluid bag being near-empty or empty. The display interface screen 124 also displays information related to infusion line management. The display interface screen 124 may include a touch interface configured to receive touch gestures that are related to available displayed options or features.

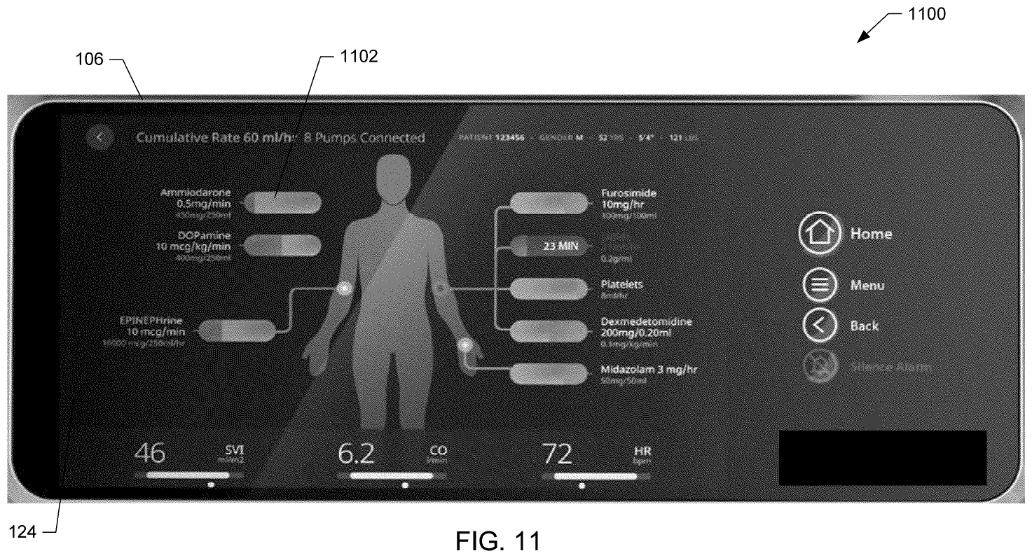

The memory device 122 may also be configured to store a data structure or file 127 that relates infusion pumps 102 to respective graphical icons that are displayed within an infusion line mapping interface 1100, as shown in FIG. 11. The data structure or file 127 may include an index that associates an infusion pump identifier or hardware address with an assigned graphical icon. As discussed in more detail below, the association occurs after a new infusion pump is added and compatibility checks are performed. The data structure or file 127 may also store at least some infusion therapy progress data 116 including a fluid type/name, an infusion rate, a volume to be infused, and/or a volume of fluid remaining within a fluid container. The application 126 is configured to obtain the infusion therapy progress data 116 from a patient's EMR, the gateway server 114, and/or receive the data 116 directly from the infusion pump 102 when connected to a hub device 400 (discussed below in more detail).

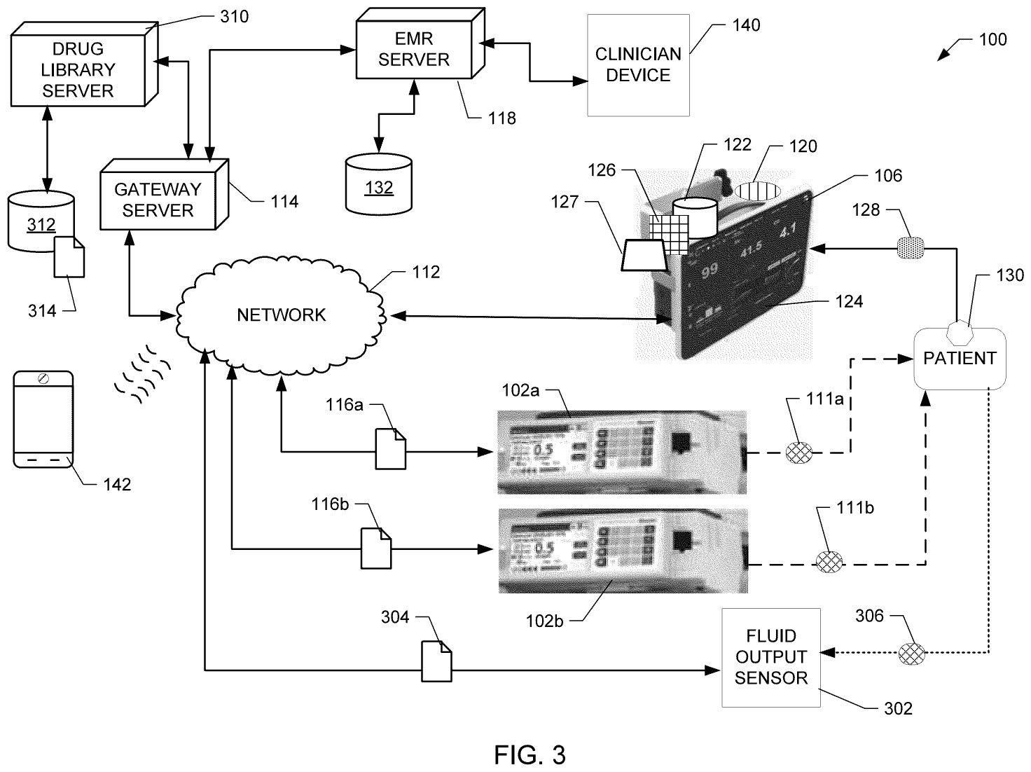

The hemodynamic management apparatus 106 is communicatively coupled to the gateway server 114 via the network 112. While FIG. 1 shows a wireless link, in other embodiments, the hemodynamic management apparatus 106 is connected to the network 112 via a wired link, such as an Ethernet connection, as shown in FIG. 3. As discussed in more detail below, the hemodynamic management apparatus 106 communicates with the gateway server 114 to receive information for display.

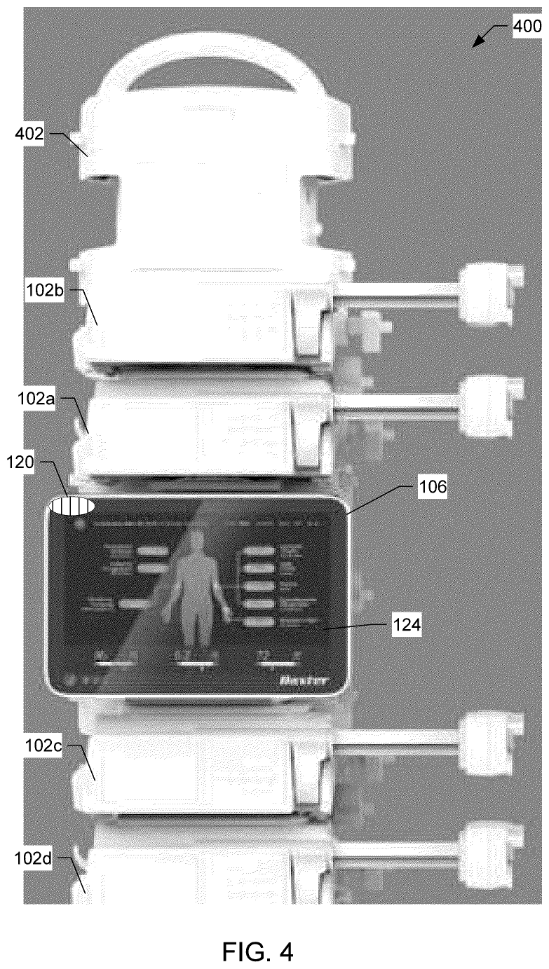

In some alternative embodiments (shown in FIGS. 4 to 6), the hemodynamic management apparatus 106 is connected to the infusion pump 102 via a hub device. The hemodynamic management apparatus 106 may communicate directly with the infusion pump 102 via the hub device without having to use the network 112. The communication may be via a universal serial bus (“USB”) connection or a controller area network (“CAN”) connection. Reference is made herein to communication between the hemodynamic management apparatus 106 and other medical devices including the infusion pump 102. It should be appreciated that the communication may occur via the network 112 and the gateway server 114. Alternatively when a hub device is used, the communication may occur locally.

As shown in FIG. 1, the hemodynamic management apparatus 106 is configured to receive physiological data 128 related to a patient from one or more sensors 130. The sensors 130 may non-invasively monitor the patient for physiological data 128 that provides for hemodynamic monitoring. The sensors 130 may include, for example, a blood pressure sensor (e.g., a blood pressure cuff), a MAP sensor, a heart rate sensor, a SpO2 sensor, and/or a body temperature sensor. In some embodiments, the sensors 130 and/or the hemodynamic management apparatus 106 are configured to use the physiological data 128 to calculate one or more hemodynamic parameters including, for example, blood flow velocity, cardiac output (“CO”), arterial pressure, peripheral, total, or systemic vascular resistance (“SVR”), turbulence, wall tension, stroke volume (“SV”), stroke volume variability (“SVV”), and/or pulse pressure variation (“PPV”). The sensors 130 and/or the hemodynamic management apparatus 106 may also be configured to use the physiological data 128 to calculate a cardia index (“CI”), a stroke volume index (“SVI”), and/or a total peripheral resistance index (“TPRI”).

While the sensors 130 are shown as being communicatively coupled to the hemodynamic management apparatus 106, in other embodiments one or more of the sensors 130 are communicatively coupled to the infusion pump 102 and/or the RFT machine 104. Additionally or alternatively, one or more of the sensors 130 are configured to communicate with the gateway server 114 via the network 112. In these other embodiments, the hemodynamic management apparatus 106 is configured to access or receive the physiological data 128 from the infusion pump 102, the RFT machine 104, and/or the gateway server 114 via the network 112.

In an example, the infusion pump 102 may be connected to a pulse oximetry sensor. The infusion pump 102 may be configured to integrate or otherwise include data from the pulse oximetry sensor into the infusion therapy progress data 116 or, alternatively, transmit the pulse oximetry data separately to the gateway server 114. Similarly, the RFT machine 104 may be communicatively coupled to a blood pressure sensor, a patient weight scale, a glucose sensor, a cardiac monitor, etc. The RFT machine 104 may be configured to integrate or otherwise include data from the sensors into the dialysis therapy progress data 119 or, alternatively, transmit the sensor data separately to the gateway server 114.

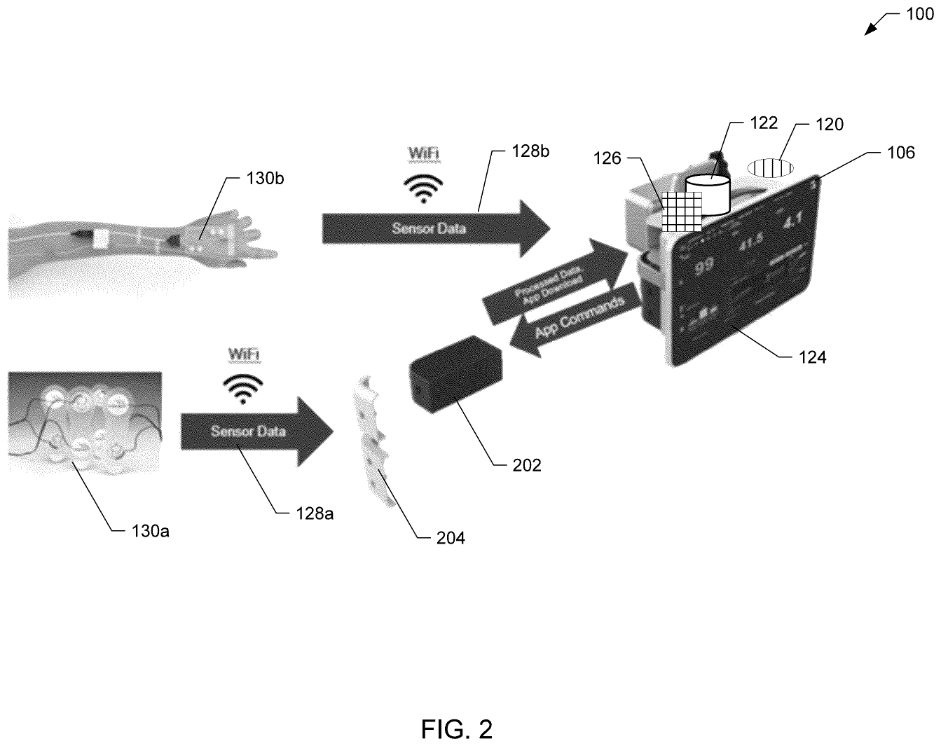

FIG. 2 is a diagram that shows one example of sensors 130a and 130b communicating directly with the hemodynamic management apparatus 106 of FIG. 1, according to an example embodiment of the present disclosure. In this example, the hemodynamic management apparatus 106 includes a sensor module 202 that is communicatively coupled to the processor 120. The sensor module 202 is configured to receive physiological data 128a from cardiac sensors 130a and convert the physiological data into hemodynamic parameters, such as SVI, TPRI, CI, CO, SV, etc. In some instances, the sensor module 202 includes an interactive interface, which is integrated with the application 126 for displaying the hemodynamic parameters. Further, the sensor module 202 may be configured to receive one or more application commands from the processor 120 for performing a hemodynamic assessment.

In an example, the sensor module 202 adds fluid responsiveness monitoring to the application 126 of the hemodynamic management apparatus 106. One or more chest patch sensors 130 are plugged into the sensor module 202, which is integrated or otherwise connected to the apparatus 106. The application 126 is configured to display an interface for monitoring stroke volume (“SV”) and cardiac output (“CO”) and to assess whether a patient will respond to additional IV fluids.

The sensor module 202 may include a Wi-Fi transceiver to enable wireless data communication with the cardiac sensors 130a. In other embodiments, the sensor module 202 and/or the hemodynamic management apparatus 106 may include a Bluetooth® transceiver. Additionally, the sensor module 202 may be operable with a communication interface 204, which is configured with a USB port, a micro-USB port, an HDMI port, or other hardwire port for the cardiac sensors 130a.

FIG. 2 also shows that the hemodynamic management apparatus 106 may communicate with an infiltration sensor 130b via a wired or wireless connection. The infiltration sensor 130b transmits physiological data 128b, which is indicative of infiltration where fluid leaks out of a vein into surrounding tissue. A transceiver at the hemodynamic management apparatus 106 is configured to receive the physiological data 128b for determining when to activate an alert or an alarm that is indicative of a detection of an infiltration. In some embodiments, the hemodynamic management apparatus 106 enables a clinician to specify which IV lines are associated with which infiltration sensors 130b such that the proper line is highlighted on the display interface screen 124 when an infiltration is detected.

Returning to FIG. 1, the gateway server 114 includes a controller, processor, router, switch, computer, etc. configured to communicate with the infusion pump 102, the RFT machine 104, and the hemodynamic management apparatus 106 via the network 112 (e.g., a wide area network, a local area network, a wireless local area network, an Ethernet, the Internet, a cellular network, or combinations thereof). The gateway server 114 may be communicatively coupled to more than one infusion pump, RFT machine, and/or hemodynamic management apparatus. Further, the gateway server 114 may communicate with other medical devices, such as physiological sensors 130. The gateway server 114 is configured to provide bi-directional communication with the infusion pump 102 for the wired/wireless secure transfer of drug libraries, infusion prescriptions, and infusion therapy progress data 116. The gateway server 114 may also be configured to integrate with a hospital information system to transmit the infusion therapy progress data 116 from the infusion pump 102 and/or the dialysis therapy progress data 119 from the RFT machine 104 to a hospital electronic medical record (“EMR”) that is managed by an EMR server 118.

The example EMR server 118 is configured to manage patients' EMRs, which are stored in an EMR database 132. The EMR server 118 receives the data 116 and 119 respectively from the medical devices 102 and 104 and uses a machine identifier and/or patient identifier associated with the data to determine a corresponding patient EMR. The EMR server 118 is configured to write the data 116 and 119 to the appropriate patient EMR within the database 132, thereby providing a record that is accessible to the hemodynamic management apparatus 106.

Further, the EMR server 118 is configured to store the physiological data 128 from the one or more sensors 130 to a patient's EMR. The physiological data 128 may be received directly from the sensors 130 and/or received from one or more of the infusion pump 102, the RFT machine 104, and/or the hemodynamic management apparatus 106. The EMR server 118 may also store infusion and/or RFT prescriptions to the appropriate patient EMR.

FIG. 1 also shows a clinician device 140 connected to the EMR server 118 and another clinician device 142 communicatively coupled to the network 142. The clinician device 140 may be located behind a firewall or the gateway server 114 and access the EMR server 118 without authentication. The clinician device 142 may be located outside of a hospital network 112 and may need to authenticate to communicate with the EMR server 118, the infusion pump 102, and/or the hemodynamic management apparatus 106. The clinician devices 140 and 142 enable a clinician to enter an infusion prescription and/or a dialysis/RFT prescription, which is transmitted to the EMR server 118 for storage in a patient's EMR. The electronic prescription is also transmitted by the gateway server 114 to the appropriate medical device 102, 104.

In some embodiments, the hemodynamic management apparatus 106 is configured to provide access for the clinician device(s) 140, 142. During a connection, the hemodynamic management apparatus 106 may cause the clinician device(s) 140, 142 to display a current view shown on the display interface screen 124. Additionally or alternatively, the hemodynamic management apparatus 106 may enable the clinician device(s) 140, 142 to select a desired interface screen to, for example, view a trend of hemodynamic parameters over time, remotely perform a hemodynamic assessment, and/or view a fluid balance of a patient.

FIG. 3 is another diagram of the hemodynamic management system 100, according to another example embodiment of the present disclosure. In this example, the RFT machine 104 has been replaced with a fluid output sensor 302, which may include a urine sensor or a sensor that measures other fluid 306 drained from a patient. The fluid output sensor 302 transmits fluid output data 304 to the gateway server 114 for routing to the EMR server 118, which stores the data 304 to an EMR of a patient. The fluid output data 304 indicates, for example, a total volume of fluid removed and/or a fluid removal rate. In other examples, the fluid output sensor 302 is communicatively coupled to an infusion pump 102 and/or the hemodynamic management apparatus 106.

FIG. 3 also shows an additional infusion pump such that infusion pumps 102a and 102b are both fluidly coupled to a patient. The infusion pump 102a is configured to infuse a first fluid 111a to the patient and transmit first infusion therapy progress data 116a to the gateway server 114 and/or the hemodynamic management apparatus 106. Additionally, the infusion pump 102b is configured to infuse a second fluid 111b to the patient and transmit second infusion therapy progress data 116b to the gateway server 114 and/or the hemodynamic management apparatus 106. Fluid IV lines from the infusion pumps 102a and 102b may be connected together such that only one patient access site is needed. Alternatively, the fluid IV lines from the infusion pumps 102a and 102b may be connected to separate patient access sites.

The first and second fluids 111a and 111b may be different fluids and comprise a medication or a drug, such as dopamine. The first and second fluids 111a and 111b may also include saline, platelets, lipids, a contrast agent, dialysate, and/or parenteral nutrition. It should be appreciated that the fluids 111a and 111b may include any solution that can be infused into a patient.

FIG. 3 also shows that the system 100 includes a drug library server 310 and a corresponding database 312. The drug library server 310 is configured to manage drug library files 314 that specify, for example, infusion hard and soft limits for specific fluids. A drug library file 314 is provided to the infusion pumps 102a and 102b via the drug library server 310 to ensure infusion parameters of an infusion prescription (or infusion parameters entered at the pump) are within clinically accepted limits. In some embodiments, the drug library file 314 specifies different limits based on a patient weight, gender, ethnicity, age, care area, etc.

In some embodiments, the drug library file 314 also specifies fluid type-to-fluid type incompatibilities and/or fluid type-to-access site incompatibilities. The fluid type-to-fluid type incompatibilities may specify, for example, fluids that cannot be infused into a patient during a same treatment or within a certain time period of each other. The fluid type-to-fluid type incompatibilities may also specify which fluids cannot be combined together via IV tubing upstream of an access site. For example, the drug library file 314 may specific that dopamine and sodium bicarbonate cannot be combined together for a common access site. In another example, a fluid type-to-access site incompatibility may specify that saline should not be administered to certain veins, such as finger veins. The incompatibilities may be based on critical care intravenous drug administration guides and/or Y-site IV compatibility guidelines.

In the illustrated embodiment, the drug library file 314 may also be transmitted by the drug library server 310 to the hemodynamic management apparatus 106 via the gateway server 114. Alternatively, the hemodynamic management apparatus 106 may use a known address of the drug library server 310 to obtain the drug library file 314. As discussed in more detail below, the hemodynamic management apparatus 106 is configured to use the drug library file 314 to perform fluid type-to-fluid type incompatibility checks and/or fluid type-to-access site incompatibility checks as part of infusion line management.

### Hemodynamic Management Apparatus Mounting Embodiments

As discussed above in connection with FIG. 3, the hemodynamic management apparatus 106 may physically connect to a hub device that houses one or more infusion pumps 102. FIG. 4 is a diagram of a hub device 400 configured to connect to the hemodynamic management apparatus 106 and the infusion pump 102 of FIGS. 1 to 3, according to an example embodiment of the present disclosure. In the illustrated example, the hub device 400 is a rack for supporting the hemodynamic management apparatus 106 in conjunction with infusion pumps 102a, 102b, 102c, and 102d. The hub device 400 may be configured to stand on a floor or table top. Alternatively, the hub device 400 may be connected to a patient's bed or a pole via at least one clamp.

The hub device 400 includes a communication module 402 that is communicatively coupled to the hemodynamic management apparatus 106 and the infusion pumps 102a, 102b, 102c, and 102d via a CAN connection, a USB connection, and/or a local wireless connection such as Wi-Fi or Bluetooth®. In the illustrated example, the pumps 102a to 102d are syringe pumps. In other embodiments, the hub device 400 may connect to large volume pumps, syringe pumps, or a combination of syringe pumps and large volume pumps. The hub device 400 is accordingly configured to enable different combinations of pumps 102 to be connected based on a prescribed infusion schedule for a patient. Further, while FIG. 4 shows four pumps 102a to 102d, the hub device 400 can include as few as one pump and as many as twelve pumps, for example.

The example pumps 102a to 102d are connected to the hub device 400 via respective shelves. The hemodynamic management apparatus 106 may be connected to the hub device 400 using an adaptor that is placed over one or more shelves. The adaptor is configured to slide over, for example, two shelves to provide improved support for the hemodynamic management apparatus 106. The illustrated embodiment enables a hemodynamic management apparatus 106 with a display interface screen 124 between 8 inches and 12 inches to be placed above or between a stack of infusion pumps 102. The relatively large size of the display interface screen 124 enables more data to be displayed for infusion line management and hemodynamic management.

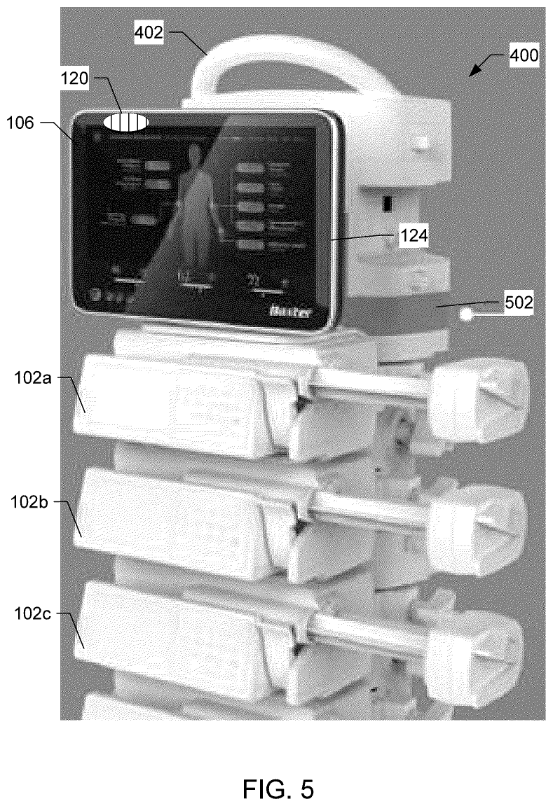

FIG. 5 is another diagram of the hub device 400 of FIG. 4 with the hemodynamic management apparatus 106 placed adjacent to the communication module 402, according to an example embodiment of the present disclosure. In this example, the communication module 402 includes an adaptor or stage 502 for receiving the hemodynamic management apparatus 106. Placement of the hemodynamic management apparatus 106 at a top of the hub device 400 enables more pumps 102a to 102c to be connected in the stack and improves visibility of the display interface screen 124.

In some embodiments, the communication module 402 is configured to provide local communication between the hemodynamic management apparatus 106 and the infusion pumps 102. The hub device 400 may include individually addressed communication ports at each shelf location. When the hemodynamic management apparatus 106 or the infusion pumps 102 are connected to the hub device 400, the communication module 402 is configured to associate a pump identifier (e.g., a media access control address (“MAC”)) with a communication port. This association enables the hub device 400 to determine which infusion pump 102 is located at which shelf or module. Further, the processor 120 of the hemodynamic management apparatus 106 is configured to register or pair with the communication module 402 using a corresponding communication port. Registration of the hemodynamic management apparatus 106 with the communication module 402 causes the communication module 402 to transmit infusion therapy progress data 116 (e.g., messages and events) that is received from the infusion pumps 102 to the hemodynamic management apparatus 106. The communicative coupling also enables the processor 120 of the hemodynamic management apparatus 106 to transmit commands, such as a command to perform a bolus, to an appropriate infusion pump 102 directly or through the communication module 402.

The example communication module 402 is also configured to be communicatively coupled to the gateway server 114 via the network 112. Infusion therapy progress data 116 from the infusion pumps 102 is transmitted to the gateway server 114 for storage in the appropriate patient's medical record within the EMR database 132. Further, the hemodynamic management apparatus 106 may communicate with the gateway server 114 via the communication module 402 of the hub device 400. In this manner, the communication module 402 is configured as a network access point and router/switch for the infusion pumps 102 and the hemodynamic management apparatus 106.

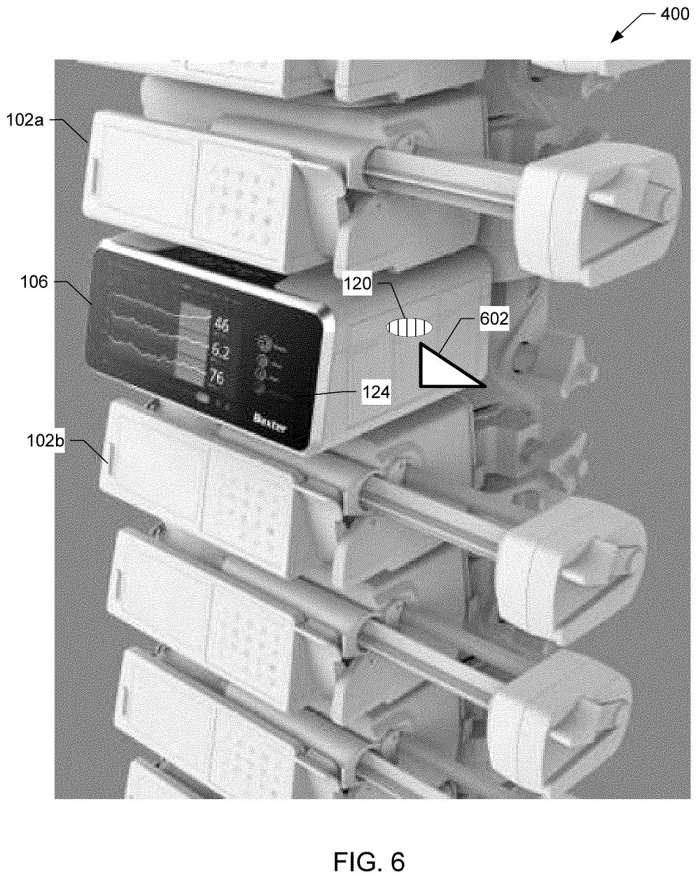

FIG. 6 is another diagram of the hub device 400 with a slim-version of the hemodynamic management apparatus 106, according to an example embodiment of the present disclosure. In the illustrated example, the hemodynamic management apparatus 106 includes a smaller display interface screen that enables the hemodynamic management apparatus 106 to fit within a shelf of the hub device 400 without needing a special adaptor. The hemodynamic management apparatus 106 is configured to occupy roughly the same space as the infusion pumps 102.

In some embodiments, the hemodynamic management apparatus 106 includes a wireless transceiver 602 that is in communication with the processor 120. The wireless transceiver 602 may include a Wi-Fi® transceiver, a Bluetooth® transceiver, etc. The wireless transceiver 602 is configured to communicatively couple to the clinician device(s) 140, 142, which may have a relatively larger screen. The clinician device(s) 140, 142 may communicate directly with the hemodynamic management apparatus 106 or indirectly via the network 112. Such a configuration enables the clinician device(s) 140, 142 to display one or more interfaces that are managed by the application 126 of the hemodynamic management apparatus 106. Further, the clinician device(s) 140, 142 may transmit commands, such as a command to perform a bolus infusion for a hemodynamic assessment and/or a command to remotely cause the hemodynamic management apparatus 106 to instruct the appropriate pump 102 to perform the commanded infusion. The clinician device(s) 140, 142 may also enable a clinician to remotely change infusion parameters using a line management interface or remotely silence alerts/alarms.

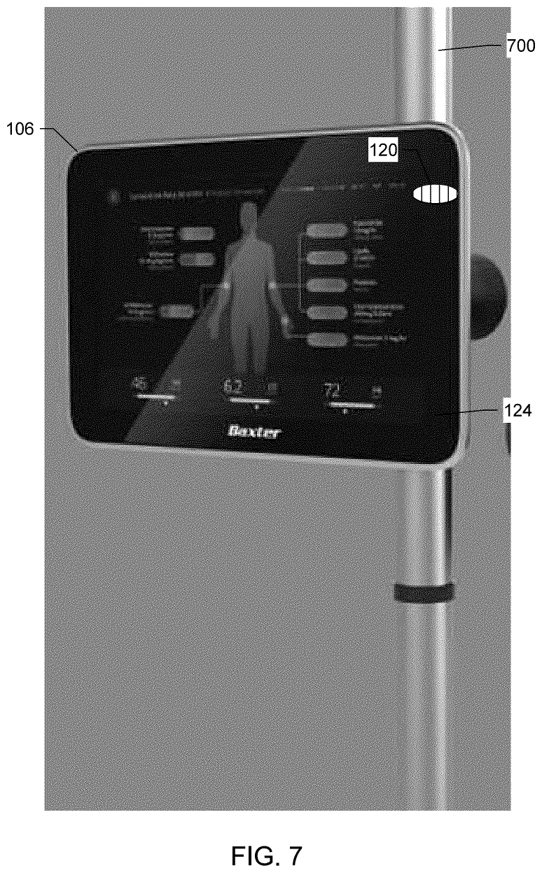

FIG. 7 is a diagram of the hemodynamic management apparatus 106 connected to a pole 700, according to an example embodiment of the present disclosure. In this embodiment, the pole 700 is configured to separately support the hemodynamic management apparatus 106. The pole 700 may be floor mounted or set on a table top. The hemodynamic management apparatus 106 may be communicatively coupled to the infusion pumps 102, the RFT machine 104, and/or the fluid output sensor 302 via the gateway server 114 and/or the network 112.

### Hemodynamic Management Apparatus Configuration Embodiments

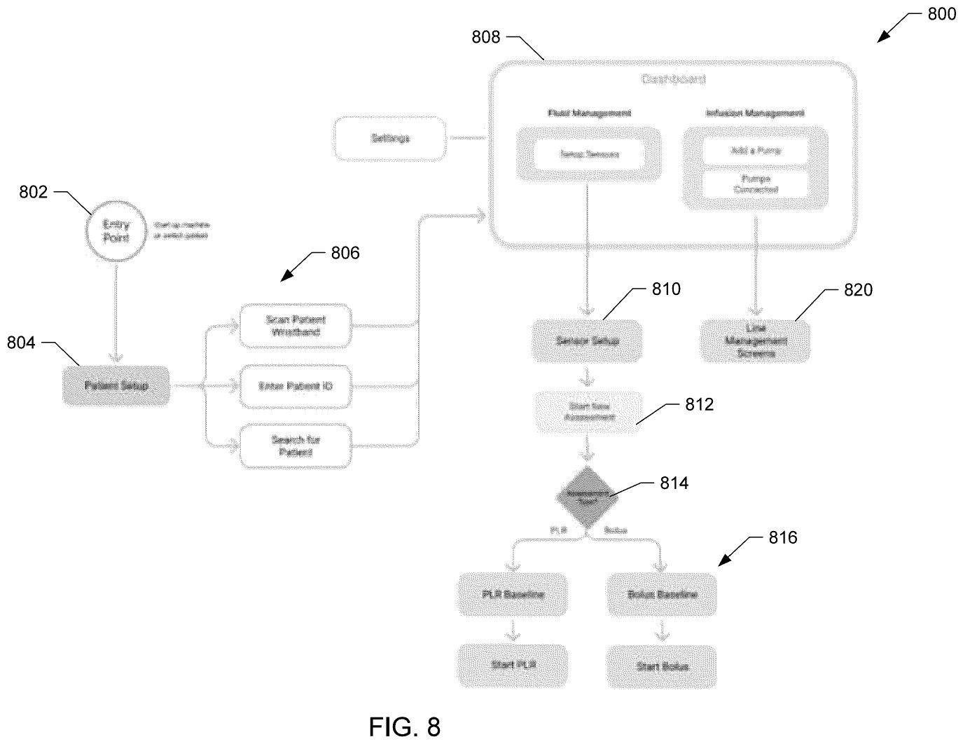

FIG. 8 shows a flow diagram illustrating an example procedure 800 to configure the hemodynamic management apparatus 106 of FIGS. 1 to 7, according to an example embodiment of the present disclosure. The example procedure 800 may be carried out by, for example, the application 126 and/or the processor 120 described in conjunction with FIGS. 1 to 3. Although the procedure 800 is described with reference to the flow diagram illustrated in FIG. 8, it should be appreciated that many other methods of performing the functions associated with the procedure 800 may be used. For example, the order of many of the blocks may be changed, certain blocks may be combined with other blocks, and many of the blocks described are optional.

The procedure 800 begins at an entry point where the hemodynamic management apparatus 106 is provisioned for a patient (block 802). This may include attaching the hemodynamic management apparatus 106 to the hub device 400 or otherwise moving the hemodynamic management apparatus 106 into a patient room. Next, the hemodynamic management apparatus 106 causes a clinician to perform a patient setup routine for patient association (block 804). This step can include using a barcode scanner to scan a patient wristband, enter a patient identifier into a registration interface of the hemodynamic management apparatus 106, or use a search interface of the application 126 to search for a patient within the EMR database 132 (block 806).

For the barcode scanning embodiment, a bar code scanner may be connected to the hemodynamic management apparatus 106. In this instance, the hemodynamic management apparatus 106 prompts a patient to scan a patient barcode or wristband, which causes the processor 120 to associate the scanned patient identifier with the hemodynamic management apparatus 106. Alternatively, the barcode scanner may be attached to a separate computer-on-wheels or other device. In these embodiments, the hemodynamic management apparatus 106 is configured to display a barcode or quick-response (“QR”) on the display interface screen 124. The code is indicative of an identifier of the hemodynamic management apparatus 106. The hemodynamic management apparatus 106 may also prompt a clinician to scan the code in addition to a barcode on a patient wristband. Scanning the code on the display interface screen 124 and the patient wristband causes the computer-on-wheels or other device to write both a scanned identifier of the hemodynamic management apparatus 106 and the patient to an EMR of the patient at the EMR database 132. The processor 120 of the hemodynamic management apparatus 106 then reads the EMRs at the database 132 searching for its identifier. After the processor 120 locates the identifier of the hemodynamic management apparatus 106, the processor 120 reads the associated patient identifier. At this point, the processor 120 has associated the hemodynamic management apparatus 106 with a patient and determined an EMR to which data 116 and/or 119 is to be written by the medical devices 102 and 104.

It should be appreciated that patient identification is not necessarily needed when the hemodynamic management apparatus 106 is connected to the same hub device 400 as the infusion pumps 102. In this instance, the infusion therapy progress data 116 transmitted by the infusion pumps 102 within the hub device 400 is automatically routed to the hemodynamic management apparatus 106 without relying on patient identification matching. Instead, the common connectivity to the hub device 400 indicates that the hemodynamic management apparatus 106 and the infusion pumps 102 are associated with the same patient. However, patient identification may still be needed to enable the hemodynamic management apparatus 106 to write to a patient's EMR in the database 132.

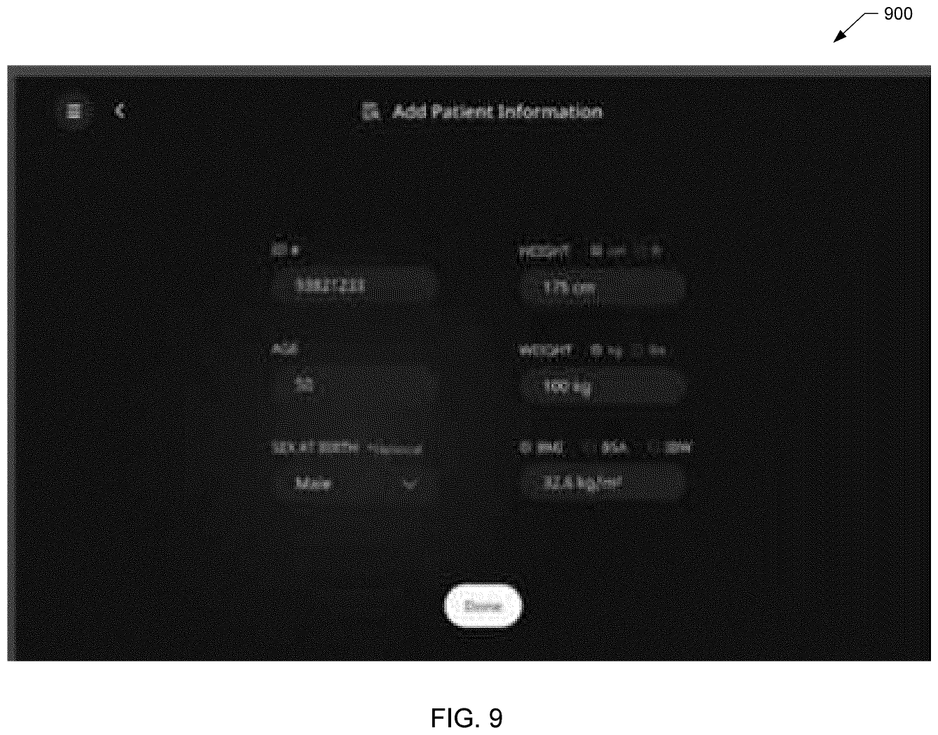

In an alternative embodiment, the hemodynamic management apparatus 106 is configured to display a registration interface, such as the registration interface 900 shown in FIG. 9. As illustrated, the registration interface 900 is displayed by the application 126 on the display interface screen 124 and prompts a clinician to enter a patient identifier, age, sex, height, weight, and body mass index. Entry of the patient identifier causes the application 126, or more generally the processor 120, to search the EMR database 132 for a corresponding patient EMR. When the patient identifier is not known, the registration interface 900 may include an option to search for a patient identifier within the EMR database 132 using, for example, a patient name, a room number, a birthdate, a social security number, etc.

Returning to FIG. 8, at this point the hemodynamic management apparatus 106 is associated with a particular patient. The application 126 of the hemodynamic management apparatus 106 next displays a dashboard (block 808) that enables a clinician to setup or configure the sensors 130 for fluid/hemodynamic management and/or add an infusion pump 102 for line management. When a sensor option is selected, the application 126 performs a sensor setup routine (block 810). This routine may include pairing with one or more sensors 130 discussed above. In some embodiments, the sensor module 202 is used to setup or configure the sensors 130.

After the sensors 130 are setup, the application 126 enables a clinician to start a new hemodynamic assessment (block 812). This option includes selecting an assessment type (block 814), such as a PLR assessment, a bolus assessment, or an automated bolus assessment (block 816). When the PLR assessment is selected, the application 126 determines a PLR baseline using the physiological data 128 from the sensors 130. The application 126 then prompts a clinician to start a PLR assessment and records the corresponding physiological data 128 from the sensors 130. When the bolus assessment is selected, the application 126 determines a bolus baseline using the physiological data 128 from the sensors 130. The application 126 then prompts a clinician to start a bolus assessment and records the corresponding physiological data 128 from the sensors 130. For an automatic bolus, the application 126 may automatically transmit commands to an appropriate infusion pump 102 with instructions and parameters for performing the bolus.

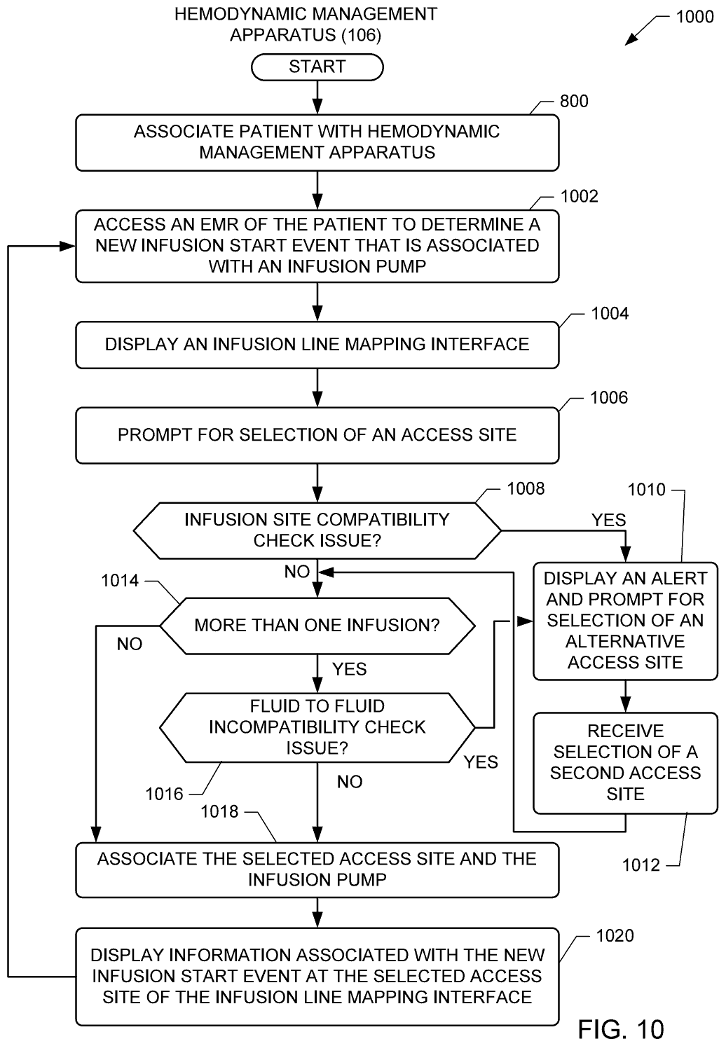

Returning to block 808, when a clinician selects the infusion management option, the application 126 is configured to display a line management interface, which enables a clinician to add a pump. This operation includes displaying one or more line management interfaces (block 820) and associating at least some of the infusion therapy progress data 116 with a designated pump graphic or icon. A procedure for adding an infusion pump is discussed in more detail in connection with FIG. 10. After configuration of the hemodynamic management apparatus 106, the example procedure 800 ends.

In some embodiments, the application is configured to add infusion pumps automatically when the hemodynamic management apparatus 106 and the infusion pumps 102 are connected to the hub device 400. In these embodiments, as each infusion pump 102 is programmed, the pump transmits infusion therapy progress data 116 (including a start event) to the hub device 400, which is routed to the processor 120 of the hemodynamic management apparatus 106. The application 126 uses at least some of the information within the infusion therapy progress data 116 to create a graphic or icon for the infusion pump 102 that shows, for example, a fluid name/type, an infusion rate, a dose, and/or information indicative of a remaining volume to be infused or an estimated remaining time until a fluid container is empty. In this embodiment, the application 126 only prompts a clinician to specify an access site on a patient. Alternatively, the application 126 also determines the access site information automatically when the infusion pump 102 includes an access site programming parameter, which may be included within the infusion therapy progress data 116.

In some instances of these embodiments, the application 126 is configured to arrange a graphic display of the infusion pumps within a line management interface based on a stack location within the hub device 400. In these instances, the communication module 402 may indicate a shelf position or stack height for each infusion pump 102 based on a known communication port to which each pump is connected. Such a configuration provides a graphic layout of infusion pumps 102 that matches the actual position within the hub device 400, which makes it easier for a clinician to quickly identify a certain infusion pump 102, such as an infusion pump that is associated with an alarm or an alert. In these examples, the display interface screen 124 may show infusion pump (or container) icons relative to a graphic of the hub device 400 or relative to each other based on a location within the hub device 400.

FIG. 10 shows a flow diagram illustrating an example procedure 1000 for infusion line management performed by the hemodynamic management apparatus 106 of FIGS. 1 to 7, according to an example embodiment of the present disclosure. The example procedure 1000 may be carried out by, for example, the application 126 and/or the processor 120 described in conjunction with FIGS. 1 to 3. Although the procedure 1000 is described with reference to the flow diagram illustrated in FIG. 10, it should be appreciated that many other methods of performing the functions associated with the procedure 1000 may be used. For example, the order of many of the blocks may be changed, certain blocks may be combined with other blocks, and many of the blocks described are optional.

The example procedure 1000 begins when the hemodynamic management apparatus 106 is associated with a patient, as described in conjunction with the procedure 800 of FIG. 8. At this point, a clinician may setup an infusion pump 102 to perform an infusion therapy. The setup includes ordering a fluid via a patient's EMR within the database 132, causing the order to be fulfilled, and causing a fluid container associated with the order to be hung adjacent to an infusion pump 102 within a patient's room. The setup also includes connecting IV tubing or an IV line set to the fluid container and priming the IV tubing or IV line set. Next, the IV line set or IV tubing is loaded into an infusion pump 102 and connected to an access site on a patient. A clinician then programs an infusion pump 102 to administer a treatment. Programming can include manually entering infusion parameters, such as a fluid name, a dose, a volume to be infused, and/or an infusion rate into the interface(s) 108, 110 at the infusion pump 102. Programming can also include scanning a label or barcode on a fluid container, which includes programming parameters that are automatically programmed into an infusion pump. Programming may further include scanning a pump barcode and/or a patient barcode, causing an electronic prescription in the patient's EMR to be transmitted to the infusion pump 102. After programming, the infusion pump 102 compares the programmed parameters to limits in a drug library file 314 and generates an alert/alarm when at least one limit is exceeded. In some embodiments, after programming, the infusion pump 102 transmits infusion therapy progress data 116 including a new infusion start event to the EMR server 118 for storage in the patient's EMR. The new infusion start event may include information indicative of an infusion pump identifier, an infused fluid name, an infusion rate, a volume to be infused, a dose, a volume remaining, and/or a time the new infusion start event was generated by the infusion pump 102.

The procedure 1000 continues by the hemodynamic management apparatus 106 next using the patient association to access an EMR of the patient to determine that a new infusion start event has occurred (block 1002). The hemodynamic management apparatus 106 may additionally be configured to scan the patient's EMR or listen via the hub device 400 for new start events. The hemodynamic management apparatus 106 may use a timestamp of the event to determine the event is new and that an infusion pump 102 is ready to begin (or has begun) an infusion. Alternatively, the new infusion start event may be transmitted via the hub device 400 to the hemodynamic management apparatus 106.

The hemodynamic management apparatus 106 next displays an infusion line mapping interface (block 1004). FIG. 11 is a diagram of an example infusion line mapping interface 1100 that may be displayed by the display interface screen 124 of the hemodynamic management apparatus 106. The infusion line mapping interface 1100 displays a graphical illustration of a human body in conjunction with graphical illustrations or icons 1102 of fluids infused into a patient via line sets. Each icon 1102 is indicative of a remaining volume of fluid within a fluid container or an estimated time remaining until a fluid container is empty, as reported by the corresponding infusion pump 102 via the infusion therapy progress data 116. Each icon 1102 also includes a fluid name, an infusion rate, and a dose. Each icon 1102 may be associated with a corresponding infusion pump identifier.

In the illustration example, the icon 1102 is created for display after the hemodynamic management apparatus 106 detects the new infusion start event. As specified in FIG. 10, the infusion line mapping interface 1100 prompts a clinician to specify an access site for the infusion (block 1006). This may include displaying a drop-down list of possible access sites or displaying possible access sites for selection on the graphical illustration of the human within the interface 1100.

After receiving a selection of an access site, the hemodynamic management apparatus 106 is configured to perform an infusion site compatibility check (block 1008). This may include using a fluid type-access site listing within a drug library file 314 to determine whether the specified fluid type is permitted to be infused into a patient at a selected access site. In some embodiments, the hemodynamic management apparatus 106 proactively performs the compatibility assessment by filtering possible access sites for selection based on the known fluid type using a fluid type-access site compatibility check within the drug library file 314.

As shown in FIG. 10, when there is a compatibility issue, the hemodynamic management apparatus 106 displays an alert and prompts a clinician for a selection of an alternative access site within the infusion line mapping interface 1100 (block 1010). The hemodynamic management apparatus 106 then receives a selection of a second, different access site (block 1012). The hemodynamic management apparatus 106 may perform another compatibility check for the second access site.

The hemodynamic management apparatus 106 also determines when more than one fluid is infused into an access site (block 1014). As shown in FIG. 11, furosimide, lipids, platelets, dexmedetomedine, and midazolam are fluidly connected to the same access site. To make the fluid connection, a clinician may use a series of Y-connectors or T-connectors. If there is more than one fluid being infused into an access site, the hemodynamic management apparatus 106 is configured to perform a fluid type-to-fluid type incompatibility check (block 1016). It should be appreciated that the incompatibility check determines whether certain fluid types can be mixed before infusion into a patient. The drug library file 314 also provides general fluid type incompatible listings that specify which fluid types cannot be infused into a patient at a same time or within a certain time duration regardless of the use of different access sites. It should be appreciated that the hemodynamic management apparatus 106 is configured to perform both an access site-specific compatibility check and a general fluid check.

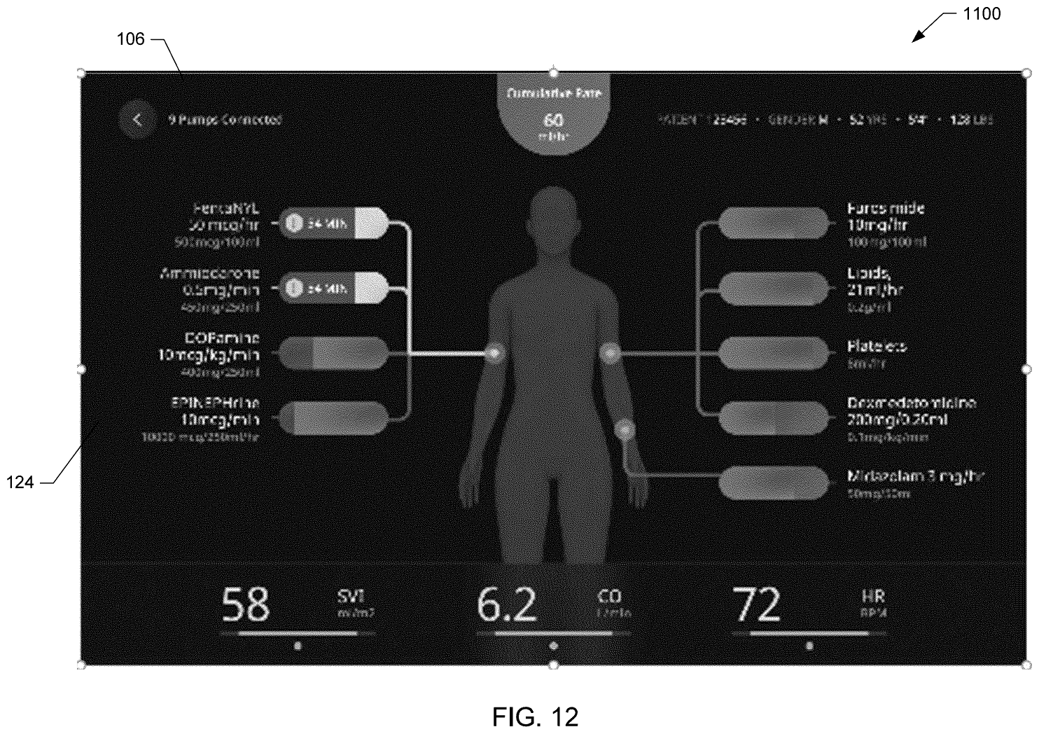

To check the access site for the combinability of multiple fluids, the hemodynamic management apparatus 106 is configured to determine the other fluid types that are associated with the given access site. The hemodynamic management apparatus 106 then determines whether the other determined fluid types are listed in an incompatibility section for the given fluid type within the drug library file 314. When there is an access site specific combinability issue, the hemodynamic management apparatus 106 displays an alert and prompts a clinician for a selection of an alternative access site within the infusion line mapping interface 1100 (block 1010). The alert may include showing incompatible infusions in red or yellow icons 1102, as shown in FIG. 12. The hemodynamic management apparatus 106 then receives a selection of a second, different access site (block 1012). In some embodiments, the hemodynamic management apparatus 106 may proactively determine access sites that are compatible with the new fluid type and provide a recommendation via the infusion line mapping interface 1100. In some embodiments, the hemodynamic management apparatus 106 is configured to first search for access sites that already have an infusion connection. When a compatible currently-in-use access site is not found, the hemodynamic management apparatus 106 is configured to identify an access site that needs a new needle access. The hemodynamic management apparatus 106 may perform another compatibility check for a user-selected second access site when other fluid types are being infused into the second access site.

To perform the general fluid compatibility specific check, the hemodynamic management apparatus 106 is configured to determine the other fluid types that are specified within the infusion line mapping interface 1100. The hemodynamic management apparatus 106 may also check a patient's EMR for past infusions. The hemodynamic management apparatus 106 then determines whether the other determined fluid types are listed in an incompatibility section for the given fluid type within the drug library file 314. When there is a general fluid type compatibility issue, the hemodynamic management apparatus 106 is configured to display an alert indicating that the selected fluid type cannot be administered due to other fluids that are being administered. The alert may indicate the specific fluids that cause the incompatibility for the patient. The hemodynamic management apparatus 106 may also specify why there is an incompatibility. In some instances, the hemodynamic management apparatus 106 transmits the alert or alarm to the corresponding infusion pump 102, thereby preventing the infusion pump 102 from performing the treatment. Further, the hemodynamic management apparatus 106 may transmit the alert or the alarm to the clinician device(s) 140, 142.

As shown in FIG. 10, the hemodynamic management apparatus 106 is configured to associate a selected access site within a given infusion pump when the compatibility checks are indicative of no incompatibility issues (block 1018). The hemodynamic management apparatus 106 may update a table or other data structure (e.g., the data structure 127) that associates access sites with icons 1102 and infusion pumps. The table or data structure 127 may be stored in the memory device 122 of the hemodynamic management apparatus 106, and provides a correspondence between infusions, fluid types, and access sites. After the association, the hemodynamic management apparatus 106 displays information associated with the new infusion start event at the selected access site of the infusion line mapping interface 1100 (block 1020). As shown in FIG. 11, this includes displaying the icon 1102 with information indicative of remaining amount of fluid in a fluid container, a graphic showing a connection between the icon 1102 and an access site, a fluid type name, an infusion rate, and a dose. In some embodiments, an identifier of the infusion pump may also be displayed. When a given fluid type is connected to other fluid types for an access site, the infusion line mapping interface 1100 shows the infusion lines as being connected together before a single line to the access site. The clinician may then press a ‘start’ button on the newly added infusion pump to begin the treatment. Alternatively, the hemodynamic management apparatus 106 may transmit a message to the newly added infusion pump 102 indicating it can start the infusion treatment. Returning to FIG. 10, the example procedure 1000 continues when another infusion start event is detected by or otherwise received in the hemodynamic management apparatus 106.

In some embodiments, the infusion line mapping interface 1100 enables a clinician to quickly identify which infusion pump is associated with each infusion that is represented by the respective graphical icon. For example, selection of a graphical icon 1102 may cause the application 126 to display additional information about the infusion pump 102 and/or the infusion. The additional information can include an infusion pump identifier, a volume to be infused, an infusion rate, and/or an infused fluid name. The additional information may also include an option to ping the infusion pump 102. Selection of the ping option causes the application 126 to transmit a message to the infusion pump 102 that causes the infusion pump 102 to emit a sound, blink the display screen 108, or otherwise display a graphical/visual indication that the pump was selected. The message may be sent directly to the infusion pump via the hub device 400 or transmitted to the infusion pump 102 via the gateway server 114.

FIG. 11 also shows that the application 126 may be configured to calculate a cumulative infusion rate. The application 126 sums each of the infusion rates shown within the interface 1100. The cumulative infusion rate is then displayed and may be indicative of a total fluid intake for the patient. The interface 1100 also includes hemodynamic parameters including SVI, CO, and heart rate. The parameters may be determined from physiological data 128 that is transmitted by the one or more sensors 130 that are coupled to the hemodynamic management apparatus 106. The infusion line mapping interface 1100 accordingly provides for infusion line management in addition to displaying information indicative of fluid balance or intake and hemodynamic information.

### Example Hemodynamic Modalities

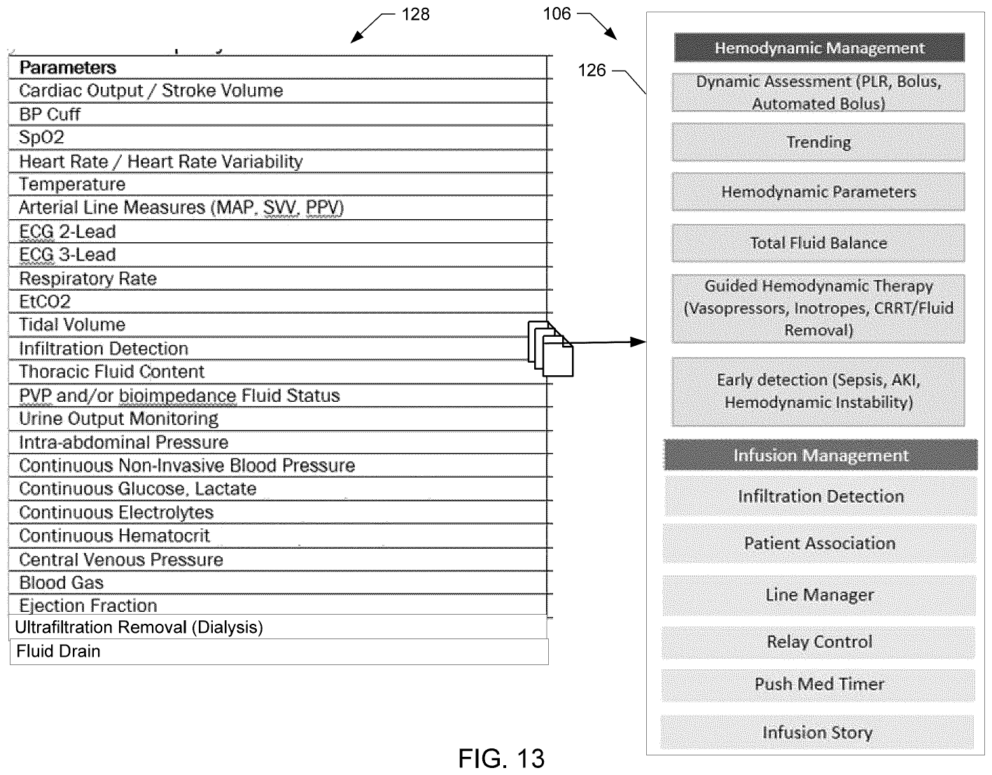

FIG. 13 is a diagram that shows different types of physiological data 128 that is input into the hemodynamic management apparatus 106 of FIGS. 1 to 7, according to an example embodiment of the present disclosure. The physiological data 128 corresponds to hemodynamic parameters that are displayed by the hemodynamic management apparatus 106. In some embodiments, the physiological data 128 is processed by the hemodynamic management apparatus 106 to determine other hemodynamic parameters such as CI, SVI, and/or TPRI.

The physiological data 128 may be received from one or more of the sensors 130. As illustrated, the physiological data 128 may include a cardiac output, a stroke volume, a blood pressure cuff measurement, a SpO2 value, a heart rate, a heart rate variability, a patient temperature, arterial line measurements including MAP, SVV, and/or PPV, an ECG 2-lead measurement, an ECG 3-lead measurement, a respiratory rate, an EtCO2 measurement, a tidal volume, an infiltration detection signal, a thoracic fluid content measurement, a PVP and/or bioimpedance fluid status, a urine output measurement, an intra-abdominal pressure value, a continuous non-invasive blood pressure, a continuous glucose and/or lactate measurement, a continuous electrolyte measurement, a continuous hematocrit measurement, a central venous pressure measurement, a blood gas measurement, an ejection fraction measurement, an ultrafiltration measurement or estimate, and/or a fluid drain measurement.

The example hemodynamic management apparatus 106 is configured to use the physiological data 128 within one or more hemodynamic modalities provided by the application 126. As shown in FIG. 12, the modalities can include a hemodynamic assessment comprising a PLR, a bolus, and/or an automated bolus. The modalities also include hemodynamic parameter display and trending, total fluid balance management, and/or guided hemodynamic therapy related to vasopressors, inotropes, and/or CRRT fluid removal. The modalities also include providing for the early detection of sepsis, acute kidney injury (“AKI”), and hemodynamic instability. Regarding infusion management, the modalities include infiltration detection, patient association, IV line management, relay control, medication timer management, and infusion story management.

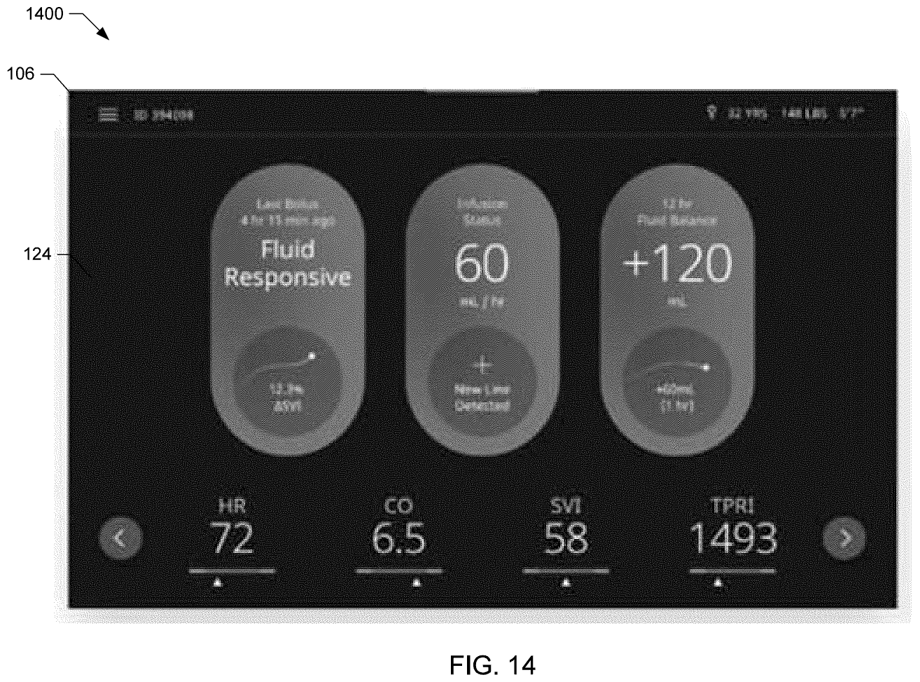

FIG. 14 is a diagram of a hemodynamic dashboard interface 1400 that is displayed on the display interface screen 124 by the application 126 of the hemodynamic management apparatus 106 of FIGS. 1 to 7, according to an example embodiment of the present disclosure. The hemodynamic dashboard interface 1400 includes a section for fluid responsiveness/assessment, infusion status, and fluid balance. The fluid responsiveness section indicates that a hemodynamic bolus assessment was last performed 4 hours and 15 minutes ago, where an ASYI has increased 12.3% since the assessment. The infusion status indicates a total fluid infusion rate in addition to an indication that a new infusion line has been detected. The fluid balance section indicates a 12-hour fluid balance of+120 milliliters (“mL”) with a recent change of+60 mL within the past hour. As discussed in more detail below, selection of a section causes the application 126 to open another interface. The hemodynamic dashboard interface 1400 also includes a section for hemodynamic parameters including a heart rate, CO, SVI, and TPRI. The SVI and TPRI parameter values may be determined from one or more computations or comparisons of the physiological data 128. It should be appreciated that in other embodiments, additional or fewer hemodynamic parameters may be displayed within the dashboard interface 1400. The hemodynamic dashboard interface 1400, and the hemodynamic management apparatus 106 more generally, accordingly integrates hemodynamic monitoring and therapy monitoring for hospital patients to deliver safer, more effective, individualized therapy during a patient's hospital stay.

### Fluid Responsiveness Embodiment

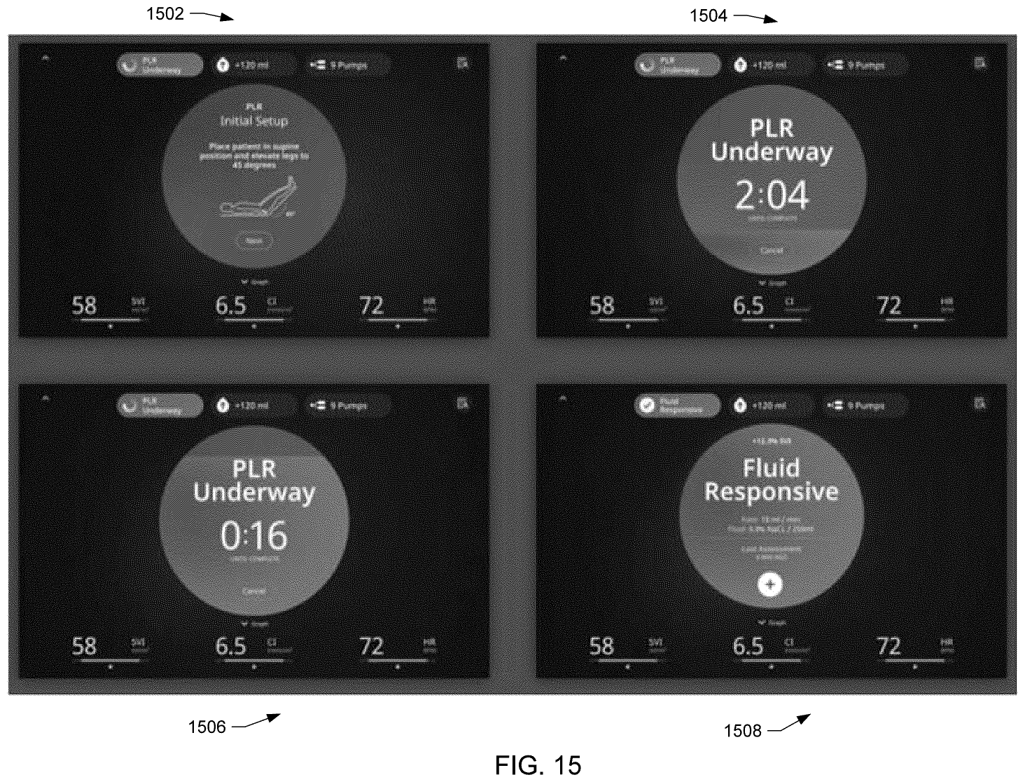

FIG. 15 shows diagrams of fluid responsiveness interfaces 1502, 1504, 1506, and 1508 that may be displayed by the application 126 of the hemodynamic management apparatus 106, according to an example embodiment of the present disclosure. The fluid responsiveness interfaces 1502, 1504, 1506, and 1508 are configured to provide a hemodynamic assessment in conjunction with displaying hemodynamic parameters including, for example, SVI, CI, and heart rate.

A first fluid responsiveness interface 1502 may be displayed after a clinician selects the fluid responsiveness section of the dashboard interface 1400 of FIG. 14 and then selects to perform a PLR assessment. The fluid responsiveness interface 1502 includes prompts for performing the PLR. After a patient is positioned, selection of the ‘Next’ option causes the application 126 to start a timer while the PLR assessment is ongoing, as shown in the fluid responsiveness interface 1504. Before the PLR assessment, the application 126 may store baseline hemodynamic parameter values, and then after the assessment begins, the application 126 aggregates or tracks the new hemodynamic parameter values. The application 126 continues the assessment, as shown in the fluid responsiveness interface 1506, until the timer expires. The application 126 then displays the fluid responsiveness interface 1508, which shows information indicative of fluid responsiveness, as determined from the measured or calculated hemodynamic parameters.

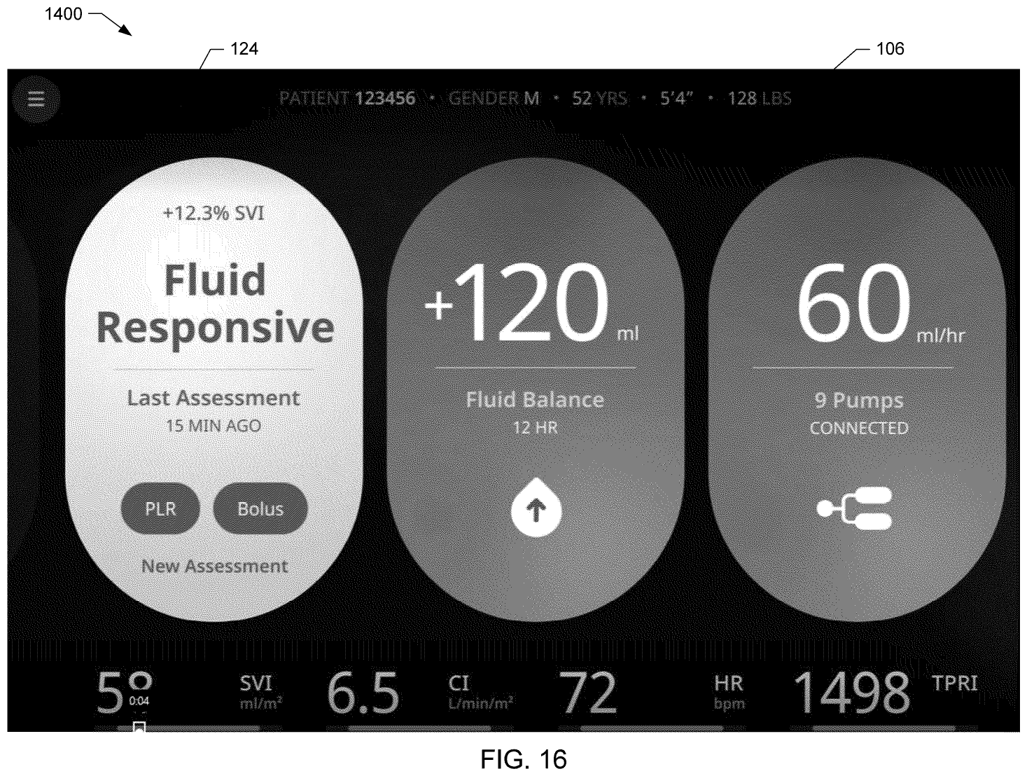

This includes, for example, a percent change for the SVI, a fluid rate, and a fluid type for the assessment. In this manner, the application 126 of the hemodynamic management apparatus 106 provides real-time clinical decision support regarding patient fluid responsiveness. FIG. 16 shows the hemodynamic dashboard interface 1400 after a hemodynamic assessment has been performed. The fluid responsiveness section indicates a time from a last assessment and options to perform a new assessment via a PLR or a bolus.

It should be appreciated that the application performs similar operations for a bolus hemodynamic assessment, for example, when the bolus option is selected in the hemodynamic dashboard interface 1400. In some instances, the application 126 includes a prompt for a clinician to indicate when a bolus has started. Alternatively, the application 126 is configured via the infusion line mapping interface 1100 and corresponding data structure to detect a bolus start event, which triggers the display of an assessment interface and hemodynamic assessment tracking. In yet an alternative embodiment, the application 126 may transmit an instruction via the hub device 400 or the gateways server 114 for a specified infusion pump 102 to administer the bolus. The instruction may identify the infusion pump, a start bolus instruction, and a time duration of the bolus. In this manner, the application 126 itself initiates the hemodynamic assessment for fluid responsiveness.

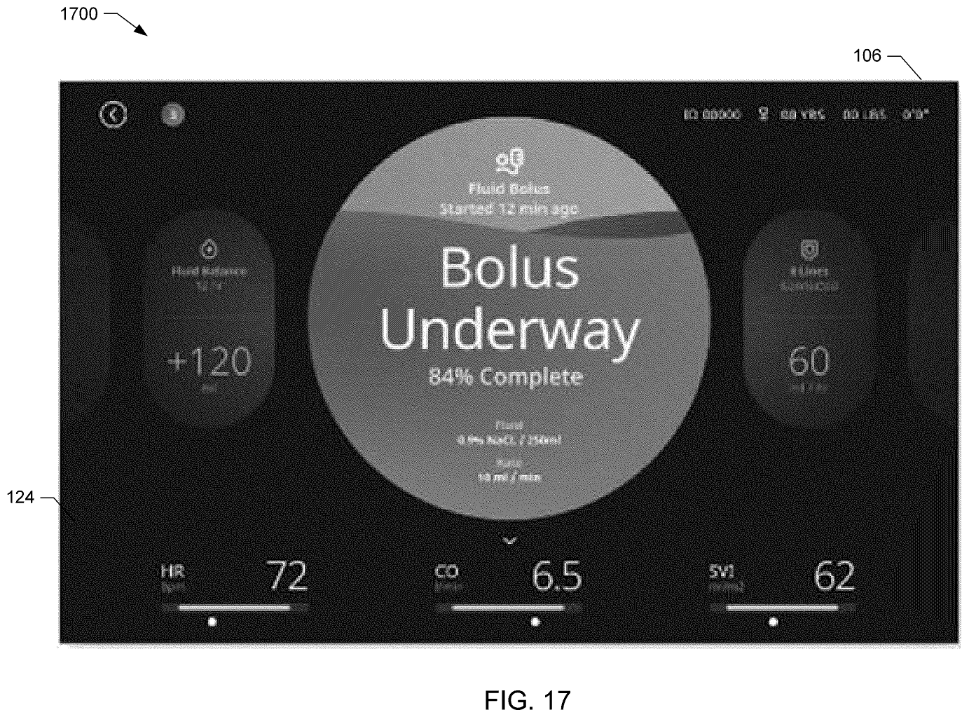

FIG. 17 is a diagram of an assessment interface 1700 that shows a status of a bolus hemodynamic assessment, according to an example embodiment of the present disclosure. The assessment interface 1700 includes a duration of the bolus, a percent complete, a fluid type, and a fluid rate. The application 126 may receive information indicative of the percent compete, fluid type, and/or rate from the infusion therapy progress data 116 generated by the corresponding infusion pump 102.

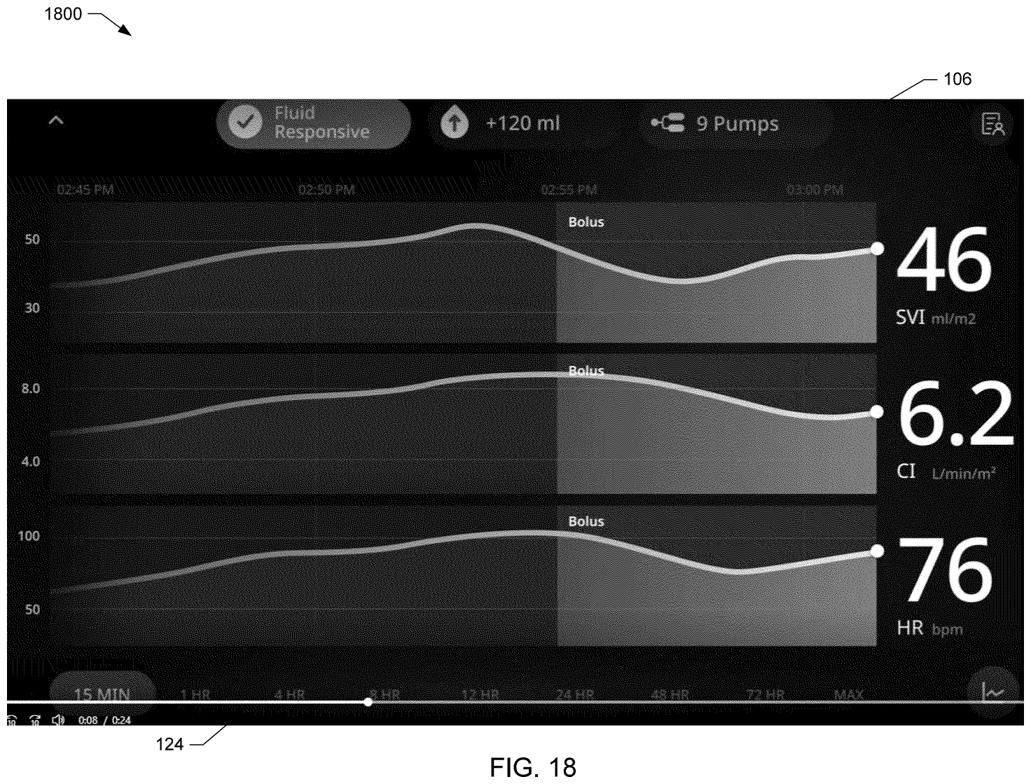

FIG. 18 is a diagram of a fluid responsiveness interface 1800 that may be shown by the application 126 of the hemodynamic management apparatus 106 after a hemodynamic assessment has occurred or is ongoing, according to an example embodiment of the present disclosure. The fluid responsiveness interface 1800 shows trends of SVI, CI, and heart rate overtime including a baseline before the hemodynamic assessment. It should be appreciated that the fluid responsiveness interface 1800 may include additional or fewer hemodynamic parameters. The fluid responsiveness interface 1800 also highlights sections of the trend data that corresponds to the hemodynamic assessment, which highlights how hemodynamic parameters have responded to the assessment. The fluid responsiveness interface 1800 further includes features to allow a clinician to change a time scale of the trended data between fifteen minutes, one hour, four hours, twelve hours, twenty-four hours, forty-eight hours, etc. The fluid responsiveness interface 1800 may accordingly provide real-time, continuous information regarding heart rate, cardiac index, cardiac output, stroke volume index, stroke volume, and/or total peripheral resistance.

Further, while FIG. 18 shows hemodynamic parameters, it should be appreciated that the application 126 is configured to provide interfaces that display values and/or physiologic trends of other physiological data 128, which may displayed in conjunction with infusion or fluid balance trends. For example, heart rate, SVI, and blood pressure may be displayed in conjunction with specific infusion rates or volumes, cumulative infused fluid rates/volumes, and/or a net fluid balance rate/volume.

### Infusion Status Embodiment

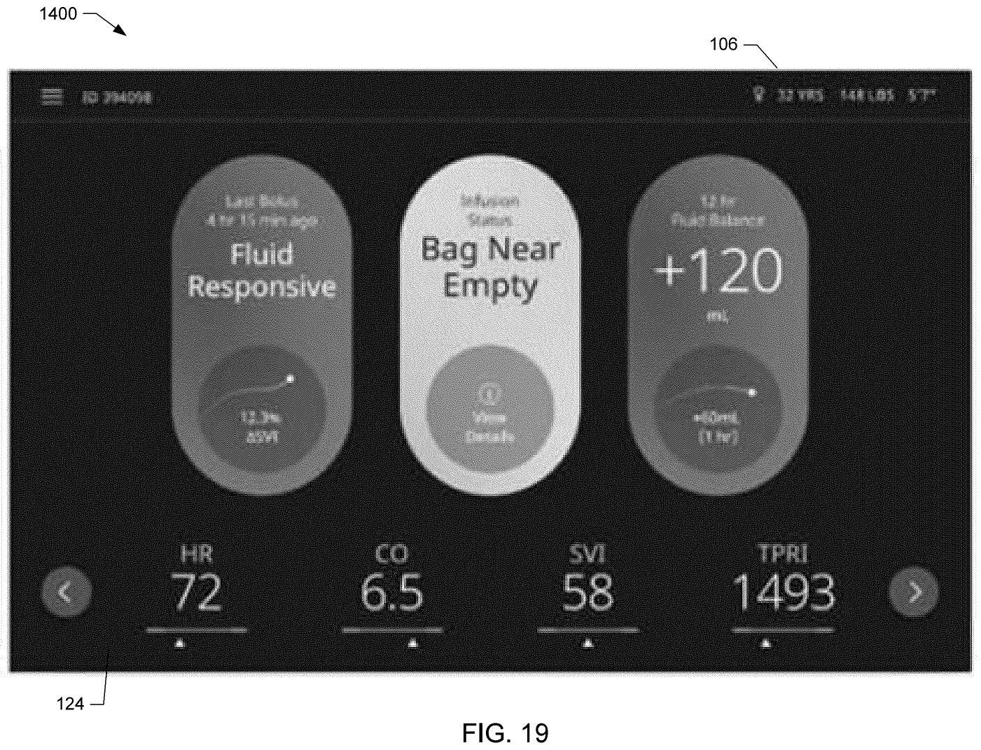

The hemodynamic dashboard interface 1400 shown in FIG. 14 also includes an infusion status section. The application 126 is configured to detect, within the infusion therapy progress data 116 or the physiological data 128, alarms and/or alerts. The alarms or alerts may be indicative of infiltration, a line occlusion, a pump or IV line leak, a bag empty or near-empty, or physiological data 128 exceeding one or more physiological limits (e.g., a heart rate exceeding 125 beats per minute). When the application 126 detects and alarm and/or alert, the application 126 is configured to update the hemodynamic dashboard interface 1400. FIG. 19 is a diagram of the hemodynamic dashboard interface 1400 with the infusion status section changed to indicate information indicative or an alarm or an alert, according to an example embodiment of the present disclosure.

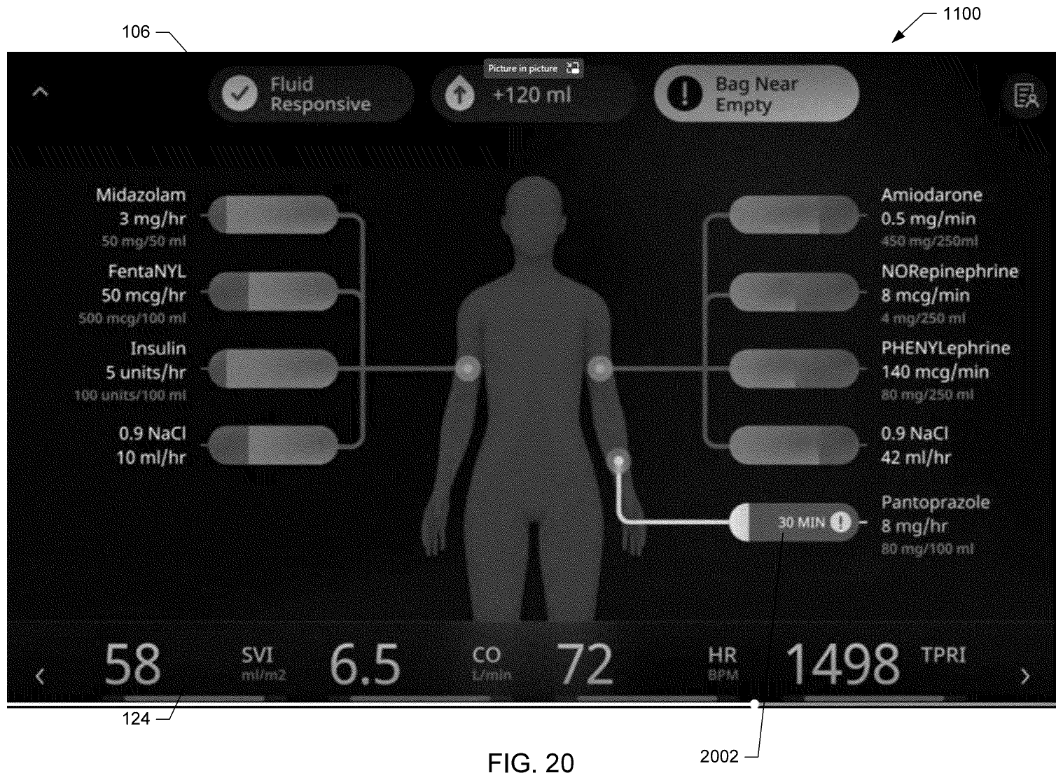

Specifically, in this embodiment, the infusion status section indicates that at least one bag or fluid container is near-empty. A clinician may select the infusion status section of the hemodynamic dashboard interface 1400, which causes the application 126 to display the infusion line mapping interface 1100, as shown in FIG. 20. The application 126 highlights an IV line and a graphical icon 2002 that are associated with the bag near-empty alert. In some embodiments, the application 126 provides the highlighting by changing the color from blue/grey to yellow. A bag empty may be shown in red. The graphical icon 2002 is also changed to show a volume of fluid that is estimated to be remaining in the fluid container or an estimated time until the fluid container is empty. The application 126 may also display a value indicative of the remaining time. Selection of the graphical icon 2002 may cause the application 126 to transmit a command to the corresponding infusion pump 202 causing it to emit an audible sound or blink a light or screen, or display some other indication as to which infusion pump 202 corresponds to the alert/alarm. In other instances, a display 108 of the infusion pump 102 may already be displaying information indicative of the alert/alarm. The alarm/alert may be removed by replacing the bag and causing a barcode scanner to scan the new bag or entering into the infusion pump 102 that a new bag has been added. Accordingly, the application 126 detects the replacement of the bag within corresponding event data of the infusion therapy progress data 116 that is stored to the patient's EMR within the database 132 or transmitted to the hemodynamic management apparatus 106 via the hub device 400.

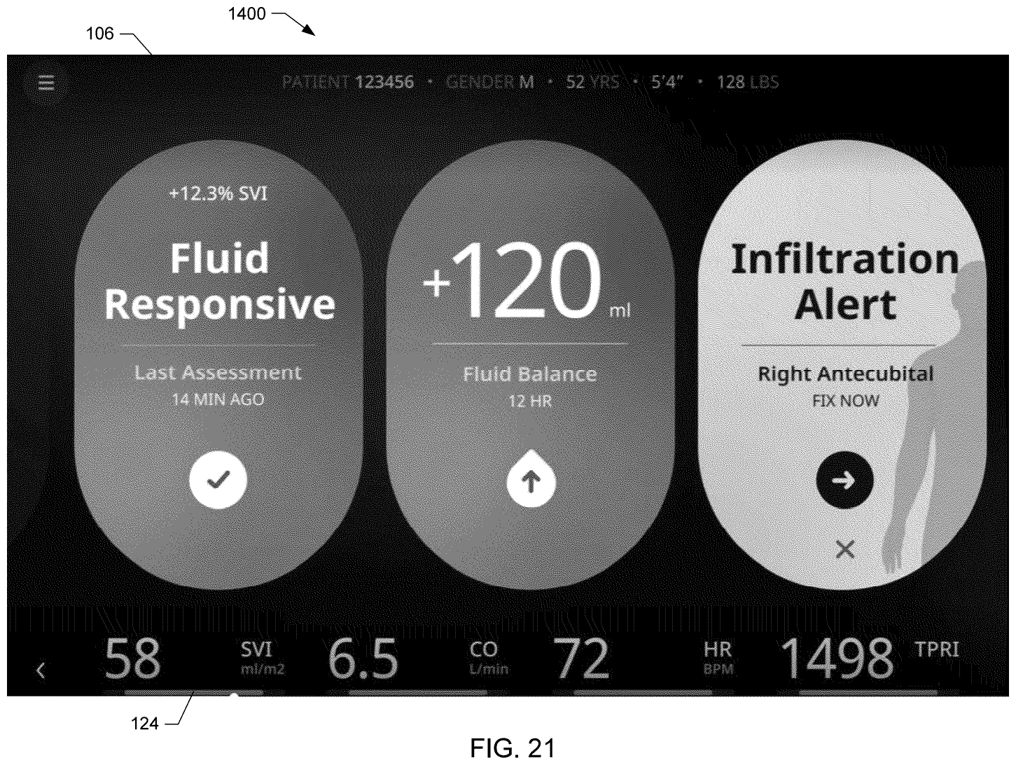

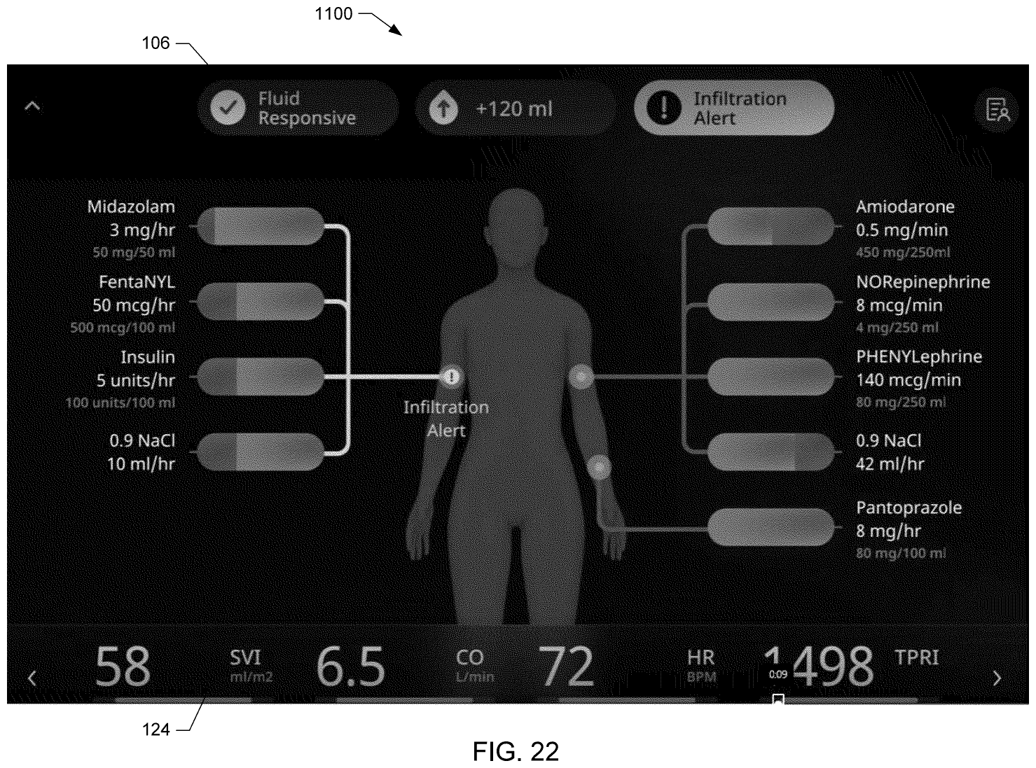

FIG. 21 is a diagram of the hemodynamic dashboard interface 1400 displaying an indication of an alert related to a detection of infiltration. The application 126 may detect infiltration based on the physiological data 128b from the sensor 130b of FIG. 2. The alert may identify the access site that is associated with the corresponding one or more infusions. In this example, the alert within the hemodynamic dashboard interface 1400 indicates that infiltration was detected at the right antecubital access site. Selection of the infusion status section of the hemodynamic dashboard interface 1400 causes the application 126 to display the infusion line mapping interface 1100, as shown in FIG. 22. Here, the application highlights the IV lines connected to the access site at issue and indicates an infiltration alert. The alert may be removed by a clinician fixing the access site connection or clearing the alert after determining infiltration is not occurring.

### Fluid Balance Embodiment

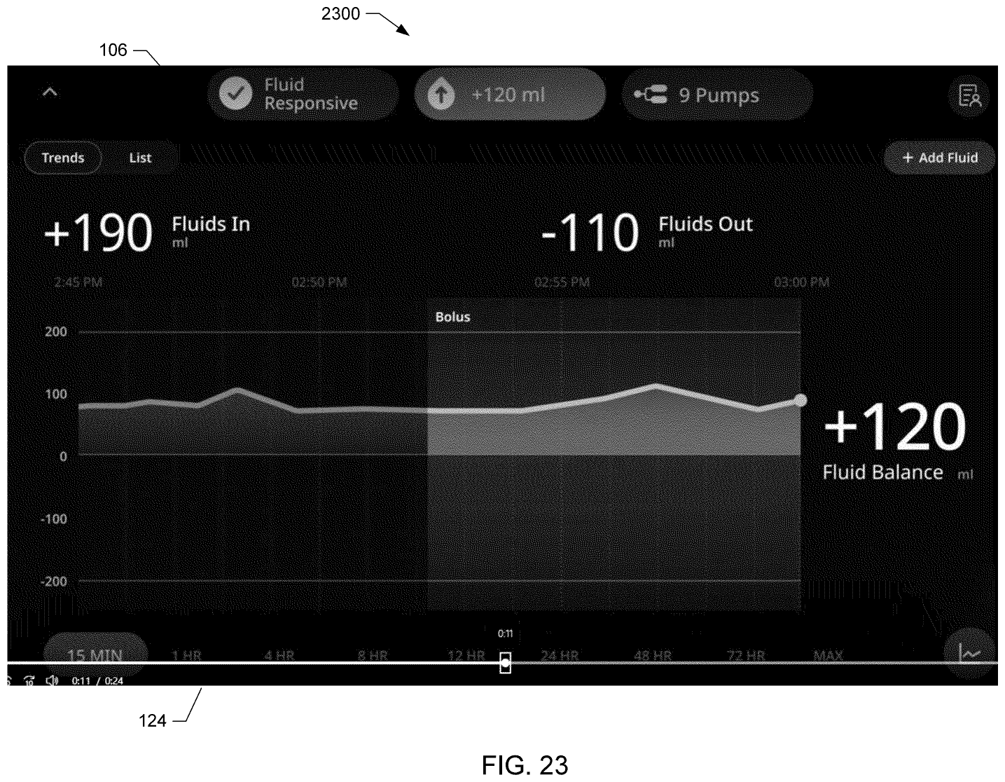

The hemodynamic dashboard interface 1400 of FIG. 14 also includes a section for fluid balance. Selection of that section causes the application 126 to display a fluid balance interface 2300, as shown in FIG. 23. The fluid balance interface 2300 provides a summary of infused fluids and detected fluids removed for a specified time period such as an hour, four hours, eight hours, twelve hours, twenty-four hours, forty-eight hours, seventy-two hours, etc. The infused fluids are determined by summing or combining the individual infusion rates shown in the infusion line mapping interface 1100. The fluids removed may be determined from the RFT machine 104, the fluid output sensor 302, and/or any other sensor 130 configured to measure fluid removed from a patient. The application 126 is also configured to determine the fluid output data from a patient's EMR within the database 132. In some embodiments, the fluid balance interface 2300 may enable a clinician to manually enter a fluid-in amount or a fluid-out amount. Further, in the illustrated embodiment, the fluid balance interface 2300 provides an indication of a fluid assessment relative to the fluid balance.

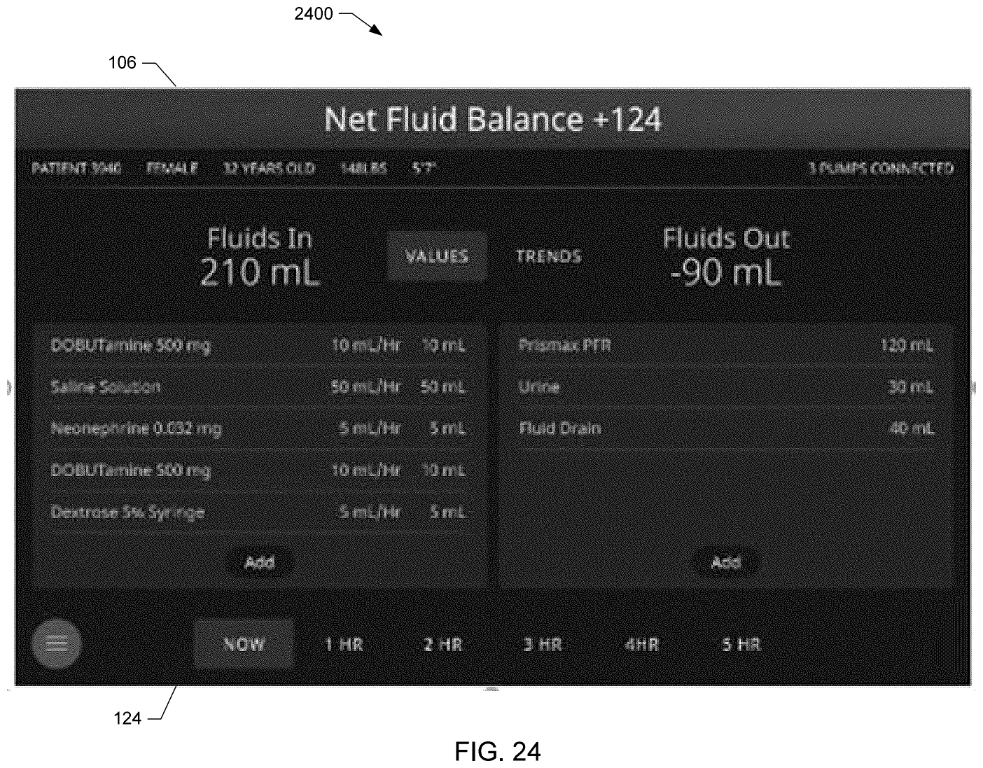

FIG. 24 is a diagram of a detailed net fluid balance interface 2400, according to an example embodiment of the present disclosure. The net fluid balance interface 2400 provides a granular list of each fluid input into a patient, as determined from the infusion line mapping interface 1100 and/or manually input by a clinician. Further, the net fluid balance interface 2400 provides a listing of fluid removed, as detected by different sources including the RFT machine 104, the fluid output sensor 302, and a drain sensor. Further, a clinician may manually enter information indicative of fluid removed. The net fluid balance interface 2400 enables the monitoring of total fluids in and total fluids out alongside current infusion information. Further, the net fluid balance interface 2400 consolidates infusion therapy information to offer a comprehensive view on one screen, thereby improving clinician experience.

### Conclusion

It will be appreciated that all of the disclosed methods and procedures described herein can be implemented using one or more computer programs or components. These components may be provided as a series of computer instructions on any conventional computer-readable medium, including RAM, ROM, flash memory, magnetic or optical disks, optical memory, or other storage media. The instructions may be configured to be executed by a processor, which when executing the series of computer instructions performs or facilitates the performance of all or part of the disclosed methods and procedures.

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims.

It should be appreciated that 35 U.S.C. 112(f) or pre-AIA 35 U.S.C 112, paragraph 6 is not intended to be invoked unless the terms “means” or “step” are explicitly recited in the claims. Accordingly, the claims are not meant to be limited to the corresponding structure, material, or actions described in the specification or equivalents thereof.

The invention is claimed as follows:

1. A hemodynamic management apparatus comprising:
a display interface screen;
a memory device storing a patient identifier; and
a processor communicatively coupled to the display interface screen and the memory device, the processor configured to:
access a patient medical record within an electronic medical record database using the patient identifier,
determine, from the patient medical record, a new infusion start event associated with an infusion pump that is fluidly connected to a patient corresponding to the patient medical record, the new infusion start event including information indicative of an infusion pump identifier, an infused fluid name, an infusion rate, a volume to be infused, a dose, a volume remaining, and a time the new infusion start event was generated by the infusion pump,
cause the display interface screen to display an infusion line mapping interface that shows a graphical illustration of a human body and potential access sites,
prompt for selection of an access site within the infusion line mapping interface,
after receiving a selection of an access site, associate within the memory device the infusion pump identifier and the selected access site, and
cause the display interface screen to display at least some of the information associated with the new infusion start event in conjunction with the selected access site shown within the infusion line mapping interface.

2. The hemodynamic management apparatus of claim 1, wherein the memory device is configured to store a drug library that specifies fluid type-to-access site type incompatibilities, and the processor is further configured to:
after receiving the selection of the access site, perform a check for a fluid type-to-access site incompatibility between the selected access site and the infused fluid name; and
when there is an incompatibility:
display an alert within the infusion line mapping interface and provide a prompt to change an access site for the infused fluid name associated with the infusion pump,
remove the alert after receiving a selection of a second access site, and
display the at least some of the information associated with the new infusion start event in conjunction with the selected second access site.

3. The hemodynamic management apparatus of claim 2, wherein the processor is further configured to:
use the drug library to determine a compatible access site based on the infused fluid name; and
cause the infusion line mapping interface to display a recommendation to fluidly connect the infusion pump to the determined, compatible access site.

4. The hemodynamic management apparatus of claim 1, wherein the processor is further configured to:
determine, from the patient medical record, a second new infusion start event associated with a second infusion pump that is fluidly connected to the patient corresponding to the patient medical record, the second new infusion start event including second information indicative of a second infusion pump identifier, a second infused fluid name, a second dose, a second infusion rate, a second volume to be infused, a second volume remaining, and a second time the second new infusion start event was generated by the second infusion pump;
cause the display interface screen to display the infusion line mapping interface;
prompt for selection of a second access site within the infusion line mapping interface; and
after receiving a selection of the second access site, cause the display interface screen to display at least some of the second information associated with the second new infusion start event in conjunction with the selected second access site.

5. The hemodynamic management apparatus of claim 4, wherein the memory device is configured to store a drug library that specifies fluid type-to-fluid type incompatibilities for access sites, and the processor is further configured to:
after receiving the selection of the second access site, perform a check for a fluid type-to-fluid type incompatibility between the infused fluid name and the second infused fluid name; and
when there is an incompatibility:
display an alert within the infusion line mapping interface and provide a prompt to change an access site for the second infused fluid name associated with the second infusion pump,
remove the alert after receiving a selection of a third access site, and
display at least some of the second information associated with the second new infusion start event in conjunction with the selected third access site.

6. The hemodynamic management apparatus of claim 1, wherein the processor is further configured to:
receive, from at least one sensor, hemodynamic information indicative of a cardiac stroke volume, a cardiac output, a cardiac index, a heart rate, a total peripheral resistance index (“TPRI”), or fluid responsiveness; and
cause at least some of the hemodynamic information to be displayed in conjunction with the infusion line mapping interface or within a separate hemodynamic interface.

7. The hemodynamic management apparatus of claim 1, wherein the processor is further configured to:
determine hemodynamic information indicative of a cardiac stroke volume, a cardiac output, a cardiac index, a heart rate, a total peripheral resistance index (“TPRI”), or fluid responsiveness; and
cause at least some of the hemodynamic information to be displayed in conjunction with the infusion line mapping interface or within a separate hemodynamic interface.

8. The hemodynamic management apparatus of claim 1, wherein the processor is further configured to:
combine the infusion rate of the infused fluid name with other infusion rates associated with the patient that are specified in the patient medical record;
determine fluid output rates that are specified in the patient medical record, the fluid output rates corresponding to at least one of dialysis, urine monitoring, or a fluid drain;
determine a fluid balance as a difference between the combined infusion rates and the combined fluid output rates; and
display at least the fluid balance within an interactive graphical interface or the infusion line mapping interface that is shown by the display interface screen.

9. The hemodynamic management apparatus of claim 1, wherein the processor is further configured to:
determine, from the patient medical record, a new infusion event that specifies a time the new infusion event was generated by the infusion pump and is indicative of at least one of a changed infusion rate, a changed volume to be infused, or a changed volume remaining; and
update the infusion line mapping interface based on information associated with the new infusion event.

10. The hemodynamic management apparatus of claim 1, wherein the processor is further configured to:
receive information indicative of an alarm event or determine, from the patient medical record, an alarm event; and
display a graphic or at least some of the information that is indicative of the alarm event,
wherein the alarm event includes at least one of information indicative of an infiltration detection, a line occlusion, or a fluid container being empty or near-empty.

11. The hemodynamic management apparatus of claim 1, wherein the infusion line mapping interface is configured to:
display the infused fluid name and the infusion rate; and
display a graphical icon that is indicative of the volume remaining or a time indicative of the volume remaining.

12. The hemodynamic management apparatus of claim 11, wherein the processor is further configured to:
determine the volume remaining or a time indicative of the volume remaining is less than a threshold;
display the volume remaining or the time indicative of the volume remaining in conjunction with the graphical icon; and
change a color of the graphical icon and the selected access site.

13. The hemodynamic management apparatus of claim 11, wherein the selection of the graphical icon causes the processor to display at least the infusion pump identifier, the volume to be infused, the infusion rate, and the infused fluid name.

14. The hemodynamic management apparatus of claim 11, wherein the selection of the graphical icon causes the processor to transmit a message causing the infusion pump to generate a sound or provide a visual indication.

15. The hemodynamic management apparatus of claim 1, wherein the patient identifier is entered into the display interface screen and stored to the memory device or determined from the patient medical record.

16. The hemodynamic management apparatus of claim 1, further comprising an adapter for connection to a hub device that is also connected to the infusion pump.

17. A hemodynamic management method comprising:
accessing, via a processor, a patient medical record within an electronic medical record database using a patient identifier;
determining, from the patient medical record using the processor, a new infusion start event associated with an infusion pump that is fluidly connected to a patient corresponding to the patient medical record, the new infusion start event including information indicative of an infusion pump identifier, an infused fluid name, an infusion rate, a volume to be infused, a dose, a volume remaining, and a time the new infusion start event was generated by the infusion pump;
causing, via the processor, a display interface screen to display an infusion line mapping interface that shows a graphical illustration of a human body and potential access sites;
prompting, via the processor, for selection of an access site within the infusion line mapping interface;
after receiving a selection of an access site, associating, via the processor, the infusion pump identifier and the selected access site; and
causing, via the processor, the display interface screen to display at least some of the information associated with the new infusion start event in conjunction with the selected access site shown within the infusion line mapping interface.

18. The hemodynamic management method of claim 17, the method further including:
after receiving the selection of the access site, performing, via the processor, a check for a fluid type-to-access site incompatibility between the selected access site and the infused fluid name using a drug library that specifies fluid type-to-access site type incompatibilities; and
when there is an incompatibility:
displaying, via the processor, an alert within the infusion line mapping interface and providing a prompt to change an access site for the infused fluid name associated with the infusion pump,
removing, via the processor, the alert after receiving a selection of a second access site, and
displaying, via the processor, the at least some of the information associated with the new infusion start event in conjunction with the selected second access site.

19. The hemodynamic management method of claim 18, the method further including:
determining, via the processor, a compatible access site based on the infused fluid name using the drug library; and
causing, via the processor, the infusion line mapping interface to display a recommendation to fluidly connect the infusion pump to the compatible access site.

20. The hemodynamic management method of claim 17, the method further including:
combining, via the processor, the infusion rate of the infused fluid name with other infusion rates associated with the patient that are specified in the patient medical record;
determining, via the processor, fluid output rates that are specified in the patient medical record, the fluid output rates corresponding to at least one of dialysis, urine monitoring, or a fluid drain;
determining, via the processor, a fluid balance as a difference between the combined infusion rates and the combined fluid output rates; and
displaying, via the processor, at least the fluid balance within an interactive graphical interface or the infusion line mapping interface that is shown by the display interface screen.
